SEQUENCE LISTING (35 sequences)

SEQ ID NO: 1 - DNA, 3849 bp, synthetic construct
  source 1..3849: mol_type = other DNA
atgactttgcaagaaacttctgttttggaaccaactttgcaaggtactactactttgccaggtttgttggctcaaagagttgctgaacacccagaagctatcgctgttgcttacagagacgacaagttgactttcagagaattggcttctagatctgctgctttggctgactacttggaacacttgggtgtttctgctgacgactgtgttggtttgttcgttgaaccatctatcgacttgatggttggtgcttggggtatcttgaacgctggtgctgcttacttgccattgtctccagaatacccagaagacagattgagatacatgatcgaaaactctgaaactaagatcatcttggctcaacaaagattggtttctagattgagagaattggctccaaaggacgttactatcgttactttgagagaatctgaagctttcgttagaccagaaggtactgaagctccagctgctagatctgctagaccagacactttggcttacgttatctacacttctggttctactggtaagccaaagggtgttatgatcgaacacagatctatcgttaaccaattgggttggttgagagaaacttacgctatcgacagatctaaggttatcttgcaaaagactccaatgtctttcgacgctgctcaatgggaaatcttgtctccagctaacggtgctactgttgttatgggtgctccaggtgtttacgctgacccagaaggtttgatcgaaactatcgttaagcacaacgttactactttgcaatgtgttccaactttgttgcaaggtttgatcgacactgaaaagttcccagaatgtgtttctttgcaacaaatcttctctggtggtgaagctttgtctagattgttggctatccaaactactcaagaaatgccaggtagagctttgatcaacgtttacggtccaactgaaactactatcaactcttcttctttcccagttgacccagctgacttggacgaaggtccacaatctatctctatcggttctccagttcacggtactacttaccacatcttggacaaggaaactttgaagccagttggtgttggtgaaatcggtgaattgtacatcggtggtatccaattggctagaggttacttgcacagagacgacttgactgctgaaagattcttggaaatcgaattggaagaaggtgctgaaccagttagattgtacaagactggtgacttgggtcaatggaacaacgacggtactgttcaattcgctggtagagctgacaaccaagttaagttgagaggttacagagttgaattggacgaaatctctttggctatcgaaaaccacgactgggttagaaacgctgctgttatcgttaagaacgacggtagaactggtttccaaaacttgatcgcttgtatcgaattgtctgaaaaggaagctgctttgatggaccaaggtaaccacggttctcaccacgcttctaagaagtctaagttgcaagttaaggctcaattgtctaacccaggtttgagagacgacgctgaattggctgctagaccagctttcgacttggaaggtgctgaaccaactccagaacaaagagctagagttttcgctagaaagacttacagattctacgaaggtggtgctgttactcaagctgacttgttgggtttgttgggtgctactgttactgctggttactctagaaaggctgctgacttggctccagctgaattgggtcaaatcttgagatggttcggtcaatacatctctgaagaaagattgttgccaaagtacggttacgcttctccaggtgctttgtacgctactcaaatgtacttcgaattggaaggtgttggtggtttgaagccaggttactactactaccaaccagttagacaccaattggttttgatctctgaaagagaagctactggtaaggctactgctcaaatccacttcatcggtaagaagtctggtatcgaaccagtttacaagaacaacatcttggaagttttggaaatcgaaactggtcacatggttggtttgttcgaacaaatcttgccagcttacggtttggacatccacgacagagcttacgaaccagctgttaaggacttgttggacgttgctgacgaagactactacttgggtactttcgaattggttccacacgctggtgctagagacgaccaagctgaagtttacgttcaaactcacggtggtaaggttgctggtttgccagaaggtcaatacagatacgaaaacggtgaattgactagattctctgacgacatcgttttgaagaagcacgttatcgctatcaaccaatctgtttaccaagctgcttctttcggtatctctgtttactctagagctgaagaagaatggttgaagtacatcactttgggtaagaagttgcaacacttgatgatgaacggtttgaacttgggtttcatgtcttctggttactcttctaagactggtaacccattgccagcttctagaagaatggacgctgttttgggtgctaacggtgttgactctgctccaatgtacttcttcgttggtggtagaatctctgacgaacaaatcggtcacgaaggtatgagagaagactctgttcacatgagaggtccagctgaattgatcagagacgacttggtttctttcttgccagactacatgatcccaaacagagttgttgttttcgacagattgccattgtctgctaacggtaagatcgacgttaaggctttggctgcttctgaccaagttaacgctgaattggttgaaagaccattcgttgctccaagaactgaaactgaaaaggaaatcgctgctgtttgggaaaaggctttgagaagagaaaacgcttctgttcaagacgacttcttcgaatctggtggtaactctttgatcgctgttggtttggttagagaattgaacgctagattgggtgtttctttgccattgcaatctgttttggaatctccaactatcgaaaagttggctagaagattggaaagagaagttgctcaagaatcttctagattcgttagattgcacgctgaaactggtaaggctagaccagttatctgttggccaggtttgggtggttacccaatgaacttgagatctttggctggtgaaatcggtttgggtagatctttctacggtgttcaatcttacggtatcaacgaaggtgaaactccatacgaaactatcactgaaatggctaagaaggacatcgaagctttgaaggaaatccaaccagctggtccatacactttgtggggttactctttcggtgctagagttgctttcgaaactgcttaccaattggaacaagctggtgaaaaggttgacaacttgttcttgatcgctccaggttctccaaaggttagagctgaaaacggtaaggtttggggtagagaagcttctttcgctaacagaggttacactactatcttgttctctgttttcactggtactatctctggtccagacttggacagatgtttggaaactgttactgacgaagcttctttcgctgaattcatctctgaattgaagggtatcgacgttgacttggctagaagaatcatctctgttgttggtcaaacttacgaattcgaatactctttccacgaattggctgaaagaactttgcaagctccaatctctatcttcaaggctgttggtgacgactactctttcttggaaaactcttctggttactctgctgaaccaccaactgttatcgacttggacgctgaccactactctttgttgagagaagacatcggtgaattggttaagcacatcagatacttgttgggtgaataa

SEQ ID NO: 2 - DNA, 4134 bp, synthetic construct
  source 1..4134: mol_type = other DNA
atgtcagttgaaacaattccttgttcacgcagagcagcattaggcctgccgggcctactgcgtgagcgtgcacgcgctacacctgatcgcacagctgctgttcatgaacatcagtcacttacatttgcgcaactgacggaggactcatcacatgttggcgctcttcttcgccaggctggcgtgggacgtgactctagagttggcgtattcatggagccttcacttgatcttcttacaggcgtttgggggatactttgggctggcggctgttatgttcctctttcacctgaatatcctgaagaacgcattgcttatatgcttgctgatgctggcgttgatatagtcctgacgcaagagtttcttcgctcaacacttcaggaacttgctcctgctggcgttgtggtgctcacccttgatgagatgttacgcacagctgaacgcgatggctcagctttcggccgtccggagccggaagtcagaccagacgacctcgcttatgttatttatacatcaggctcaacaggcaaacctaaaggcgttatggttgaacatcgctcaattgtttcacagatgcgctggcttcatgatgaatgtggcattgatgagaatgagattattcttcagaagactccgatgtcatttgatgcggcacaatgggagcttcttgctcttgcttgtggctcaacagttgttatgggctcatcaggcatttatcgcgatcctgaagctattatttcaacagttcagcgccatggcgttacaacacttcagtgtgttcctacacttcttcaggctcttcttgatacagagaagttcccggactgcggcacacttcgccgcatattctcgggtggcgaagctctttcacgctcacttgccgcgcaatgcctcgatacaatgcctggcgctcgccttgttaacctttatggccctacagaatgtacaattaacgcttcatcatttgttgttgatcgtgcggctttagaagatggtcctcttgttatgcctattggcacacctgttcatgatacatcacttcatgtcttacggccggacggtgcaccggtctcagctggcgaaattggcgaactttatattggcggcgttcaggttgctcgcggctatttgggtagaccggacctgacaggcgatcgctttatggctgatcctttctcggacgcaccgggtagtcggctttatcgcacaggcgatcttgctcatgttaacgctgatggcacagttcagttcgtaggtcgtgcagataatcaagttaaacttcgcggctatcgcgttgaactggacgagatcaggcagacagttgaaacacatgattgggttcgcgctgccgccgtactactgagggacgacgcaactaccggctttcagaacctcgtcgcgtttgtagaactcaaccctaaagaagctgctcttatggatcagggcaaccatggctcacatcatcagtcaaaggcttcgcggttgcaagtacgcgctcagcttgctcatcctggctgtcgagacgacgcggacctggctggccgcgctgctattgatcttcctggcgctgaagctacacctggccagcgcgctcttgctttctctcggaagacttaccgcttctacgagggctcacctgttacacgcgatgatattcttcatcttcttggccctcgccctcgccctcgcccttcagctcgcacatcagatatagtaggtagagatgaactgggcacaattcttcgcaactttggccgccatctttcagatcagcgccttcttcctaaatatgcttatgcttcacctggctcactttatgctacacagctttatgttgaaattggcggcggccatgatgtgccggcgggtctctactattatcatcctcttcatcatcgccttgttcttgttggccctgcttcagaaacagaaacatcacctgttcgcattcatttcctgggaaagcacggtgctattgaacctgtttatcgcaacaacgttcgcgaagttcttgaaattgaagctggccatatggttggcttgttcgaggaggttcttcctgctcatggccttcgcattgctgctgctgcttatcagcctgctgttcgccatcgccttgattgtgctcctgaagatcattatcttggctcatttgatcttcttcctcaggctcgcggcgcttcagaagatacagatacacttgatatttatgtacaagcacacagtacccgcattgaaggccttcctcctggccagtatcgctatacaggcgctggccttgttcgcattggcgatgatgttattcttaagaagcacgtgattgctattaaccagcgcgtttatgaacgctcagatttcggtatctcacttgttgctacaggctcagcttcatggcgccgctatcttgatcttggccgcggccttcagcgccttcagatgaacgatcttcatcttggctttatgtcatcaggctattcatcaaagtcgggtaacgatcttccttcagctaaacgccttggccgcattcttgctgatggcggcctacccgctgggccctcatacttctgtgtaggcggccgcgtatctgacgcacaatggcgcggcgaagatatgaaagaagatgttgttcatatgcagggccctgctgaacttattaaagaagatcttgctgctcttcttcctcgctatatgcttcctaaccgcattgttgttcttgatcgccttcctcagacagctaacggcaagatagaccttaaagctcttcagacaacacaggaagcgcaattaacggtgggagaacgcgctttcatggcgcccagaacacctcttgaacgccgcattcgcgatatttggcaggctgttcttaaacgcgatcaggtttccgtaacggacgacttctttgaactgggcggcaactcacttcttgctgttgctcttgtttcacgccttaacgctgatttcggtggcgcgattcctcttcagatcctgtttgaggcacctacagttgaacgattagcagcggcgctcgaagctacctcgccaagaccggcaagcagattagtgccacttcagcctgaaggccgcggcacacctctttattgttggcccgggttgggaggttaccctatgaaccttcgccctctcgccgccgcgttaggcacggagcgccctgttcatggcgtccaagcgcacggaataaaccctggcgaatttccttatgatgatgttcgcgctatggctgctgctgatgttgaagctattcgcgaaattcagcctcatggcccttatcttctttgtggctattcatttggcgctcgcgttgctttcgaggcagctcgccagcttgaacaggctggcgaacaggttgaacagctattcctagtggctcctggccagcctcgccttcgccctgaagatgctgttggcgctacaggtagggccgactttacggaccgcgctttcctcgcccttctcttcagcgtgttcgcaggtacactttcaggccctcgccttgatcagtgtcttcgcactgtaactgacgaggacggcttcgtggcgtttgttacagcctcatttccaggattgggtgaggagctcgtacgtgccgtcaccggcattgttcgccgcacatattcacttacatatgaatttcatgaacttcgcggccgccgccttgatgctcctgttacattagtcagggcaacggacgataactattcatttattgaacatgaaggcggctattcagctcgccctcctgctgttcatcagcttcgctcaggccattatgagctactgcgcgaacctcatgtcgctcgactcgcggccgtcctcaatgaccgcctttcagcgggtccctccacgtcaccgcggcacagtcagcctgctcaggctacagttcaggaagttggcgttcctcatattaacattaaacatttcccagtaagtattacagaagagaaggagctagaacttgttgctgctgttacaacagctgttcgcaacgctttcgggtgcacagaagaagttgtttcaattgctcttgaacctgttgctcaggaagtttggaacgaacgcgtttatattcctgaaattgttgctcgccaggagctactacgtaagactccaaactattga

SEQ ID NO: 3 - DNA, 654 bp, synthetic construct
  source 1..654: mol_type = other DNA
atgttggacgaatctttgttcccaaactctgctaagttctctttcatcaagactggtgacgctgttaacttggaccacttccaccaattgcacccattggaaaaggctttggttgctcactctgttgacatcagaaaggctgaattcggtgacgctagatggtgtgctcaccaagctttgcaagctttgggtagagactctggtgacccaatcttgagaggtgaaagaggtatgccattgtggccatcttctgtttctggttctttgactcacactgacggtttcagagctgctgttgttgctccaagattgttggttagatctatgggtttggacgctgaaccagctgaaccattgccaaaggacgttttgggttctatcgctagagttggtgaaatcccacaattgaagagattggaagaacaaggtgttcactgtgctgacagattgttgttctgtgctaaggaagctacttacaaggcttggttcccattgactcacagatggttgggtttcgaacaagctgaaatcgacttgagagacgacggtactttcgtttcttacttgttggttagaccaactccagttccattcatctctggtaagtgggttttgagagacggttacgttatcgctgctactgctgttacttaa

SEQ ID NO: 4 - DNA, 1845 bp, synthetic construct
  source 1..1845: mol_type = other DNA
atgtttgatcttgatggcacacttgttgattcacctcgcgctattgttgaagctttcgcggcggctttctgggcaatgggccttgctcctcgcgatcctgctgatattcgcgctacaattggccttcctcttgaacaggctttcgggaagctcttgggcgtgcctcagaatgacccatctgtagccgacggtgttgctcgctatcaggaagctttcaggactctcatcttaccgcgggctcattcacttgtcttcccgggagtccctgaaggccttgctgaacttcgccgccatggcgttgttgttacagttgctacatcaaagttccacgcttcagccgacgccttgttaactgccgcgggactgcgggaccatgttacaacacttgttggcgctgatgatgttacacgccctaaacctcatcctgaatcaggcttattaatattgggagaacttggcgctcgccctgaacatgctgttatggttggcgatacaacacatgatcttaagatggcggctgccgcagggctagcttcggtagccgtaacctatggcgttcatgaacgcgctgaacttgaaacagcttcacctacacatgttgctgatacatttgctcaggctgttgaacagattcttgctgttctaccagcagacggagggccggaagatacaggcacagttgaatcacttcttgatgattcaacatatcatattgaatttaacggccatcttacaaaccatattaaacatgctgttgttgctcttgctggccttggcgttgatcctggccgcattaaagcttatcatgataactatattgctcttacaccttatggctgtcgcgttgaacctgctcgccctcctcagcgccttattgatgatggcaactggcttgaacatcttggccgccgcgaagatttcgcagcctattgtgctttcttcgaccggcgcgaacgcgaacttggcatggctggccttcttcgccagtatgttcctcgccttcttgctggctgggctggcgctcttcagcatgctacaattcatcttggctgggctcttgatgctggcaaccgccgcatggctattgaaggcatagcctacctagccttcgcttatgttgattgtcatcctgaacgcgctgttccttcagaagctcctggcacagataagcccggtgactcgctgctttcaattgctcgccattgggaagagaatggacctcgccttggcgcttgggttgaagatcttgttggcacaacatcagctgatattcatcctgaacttcttcgctcaggccttcagtatcgcattgctcgcatgcttggcgaaggccatcctcttatgtatggcacaccttcatggattgctgctcaggatcctgatacatcatgggaacagctcgcgtatctcgtaaccgtgttgtatcttacagagcccggagacttcctgttacttcacttggtaacggcattacatgctatgcgccatattgcggacgcccttccagcggagcaacagcgccgcgctgttgcttgttattggacaggcatattgggagtcttattcagtcgggggcatttcgtggcaccgtctaagctgacggctattgattcattattcgacactgctcttgatgatcttgatgatcctcgctgggctcgcgaatgggattggcatattgctcgcgctgttgaagaagaagaagaacataacgctaaacttgtttatgttatgcgcgaactttggcgcggctcaggcggccgctcagtttatcgcgttgctgctggccagtttacaacaacacctgaacttcctgctacatttgaacagcctcctgttgattga

SEQ ID NO: 5 - DNA, 20 bp, synthetic construct
  source 1..20: mol_type = other DNA
gaattcgagctcggcgcgcc

SEQ ID NO: 6 - DNA, 41 bp, synthetic construct
  source 1..41: mol_type = other DNA
gctagtctcctgcaaggtcatggatcctggctgtggtgatg

SEQ ID NO: 7 - DNA, 20 bp, synthetic construct
  source 1..20: mol_type = other DNA
tgtccgggatctcgacgctc

SEQ ID NO: 8 - DNA, 20 bp, synthetic construct
  source 1..20: mol_type = other DNA
aagcattatgcggccgcaag

SEQ ID NO: 9 - DNA, 20 bp, synthetic construct
  source 1..20: mol_type = other DNA
gcgatcgctgacgtcggtac

SEQ ID NO: 10 - DNA, 20 bp, synthetic construct
  source 1..20: mol_type = other DNA
ggccggccgatatccaattg

SEQ ID NO: 11 - DNA, 20 bp, synthetic construct
  source 1..20: mol_type = other DNA
agtcgaacagaaagtaatcg

SEQ ID NO: 12 - DNA, 20 bp, synthetic construct
  source 1..20: mol_type = other DNA
gacccgtttagaggccccaa

SEQ ID NO: 13 - DNA, 21 bp, synthetic construct
  source 1..21: mol_type = other DNA
gaattcagcttggctgttttg

SEQ ID NO: 14 - DNA, 25 bp, synthetic construct
  source 1..25: mol_type = other DNA
aagcttaattaattctgtttcctgt

SEQ ID NO: 15 - DNA, 41 bp, synthetic construct
  source 1..41: mol_type = other DNA
gaaacagaattaattaagcttatgaccttgcaggagactag

SEQ ID NO: 16 - DNA, 40 bp, synthetic construct
  source 1..40: mol_type = other DNA
catctaataaccctcctcctttactcaccgagaaggtaac

SEQ ID NO: 17 - DNA, 40 bp, synthetic construct
  source 1..40: mol_type = other DNA
taaaggaggagggttattagatgctggatgagtctttgtt

SEQ ID NO: 18 - DNA, 40 bp, synthetic construct
  source 1..40: mol_type = other DNA
caaaacagccaagctgaattctcaagtcactgcagtcgca

SEQ ID NO: 19 - DNA, 20 bp, synthetic construct
  source 1..20: mol_type = other DNA
gttccctactctcgcatggg

SEQ ID NO: 20 - DNA, 20 bp, synthetic construct
  source 1..20: mol_type = other DNA
tctggataatgttttttgcg

SEQ ID NO: 21 - DNA, 41 bp, synthetic construct
  source 1..41: mol_type = other DNA
aggtatagcatgaggtcgctcggatccactagttctagagc

SEQ ID NO: 22 - DNA, 40 bp, synthetic construct
  source 1..40: mol_type = other DNA
agaaacattttgaagctatgctcgagggggggcccggtac

SEQ ID NO: 23 - DNA, 20 bp, synthetic construct
  source 1..20: mol_type = other DNA
tcccagtcacgacgttgtaa

SEQ ID NO: 24 - DNA, 20 bp, synthetic construct
  source 1..20: mol_type = other DNA
ttacgccaagcgcgcaatta

SEQ ID NO: 25 - DNA, 21 bp, synthetic construct
  source 1..21: mol_type = other DNA
tgagctcatgagcggagaacg

SEQ ID NO: 26 - DNA, 20 bp, synthetic construct
  source 1..20: mol_type = other DNA
cagtcgatcatagcacgatc

SEQ ID NO: 27 - DNA, 20 bp, synthetic construct
  source 1..20: mol_type = other DNA
gtctgacgctcagtggaacg

SEQ ID NO: 28 - DNA, 20 bp, synthetic construct
  source 1..20: mol_type = other DNA
tcatatctcattgcccccgg

SEQ ID NO: 29 - DNA, 20 bp, synthetic construct
  source 1..20: mol_type = other DNA
cctcttcgctattacgccag

SEQ ID NO: 30 - DNA, 20 bp, synthetic construct
  source 1..20: mol_type = other DNA
gagcggataacaatttcaca

SEQ ID NO: 31 - DNA, 8282 bp, synthetic construct
  source 1..8282: mol_type = other DNA
ggggaattgtgagcggataacaattcccctgtagaaataattttgtttaactttaataaggagatataccatgggcagcagccatcaccatcatcaccacagccaggatccatgaccttgcaggagactagcgtattggaaccaacccttcagggtaccactactctcccaggacttttggcccaacgggtggctgagcatccagaagccatcgcagttgcctaccgggatgataaacttactttccgtgagcttgcctcccgttcggcggcattggccgattacctggagcacctgggcgtgtccgcggacgactgtgtaggtctctttgtggaaccttcgatcgatctcatggtcggagcctggggcatcctcaacgccggcgcagcatacctgcccctcagccctgaatatcctgaggaccgtctgcgctatatgattgagaactccgagaccaaaatcattctggcgcaacagcgcctcgttagccgccttcgggaattggctcccaaggacgttacgatcgtgacgttgcgtgaatcagaggcattcgtccgccctgaaggaactgaagccccggcagcacggtcggcgcggcccgacacgctggcatatgtgatctacacctctggctccacgggaaaaccgaagggtgtaatgattgagcatcgcagcattgtaaatcaactcggctggctgcgggagacgtatgcaatcgatcgttccaaggttatcctccaaaaaactccgatgagcttcgatgcagcgcaatgggagattctgtcgccggccaacggcgcgaccgtggttatgggtgcaccgggcgtctacgctgatccagaaggcctgattgaaacgattgtgaaacataacgttactactttgcaatgtgttcctacgctcctccaaggattgattgatacggaaaaatttcctgagtgcgtttcgttgcaacaaatcttttcaggtggtgaggcattgtcgcgtctcttggcgatccagacgacgcaagaaatgccaggtcgcgcattgatcaatgtatacggaccaacggagacgaccatcaattcatcctcctttccagttgatccggcggacctggacgagggaccacagtcaatctcgattggttcacctgtacacggaaccacttatcatattctggataaagaaacgctcaagccagtaggagttggagaaattggagagttgtatatcggcggtatccaactggcgcgtggttacctccaccgtgacgacttgaccgccgagcggttcttggaaatcgagcttgaagagggagcagaaccagtgcggctctataagacgggcgacctgggccagtggaataatgatggaaccgttcagttcgcgggccgcgctgacaaccaggtgaagctgcggggctaccgcgtggagctcgatgaaatcagcttggccatcgagaaccatgattgggtccggaacgcggcagtgatcgtcaaaaatgatggacgcacgggcttccaaaatcttatcgcttgcattgaactgtcggaaaaagaggctgcccttatggaccaaggtaaccacggctcccaccatgccagcaagaagtctaagcttcaagtgaaagcgcagttgtccaatccaggtctccgcgacgatgctgaacttgcagctcgcccggcattcgaccttgaaggagccgaacccacgccggagcagcgcgctcgggtttttgcgcgtaaaacctatcggttttatgaaggcggcgccgtcacgcaggcggaccttttgggtcttctgggtgctaccgtaacggcaggttactcccgcaaagctgccgacttggccccggcagagttgggacaaatcctgcgttggtttggccagtatatctctgaagagcgcctcctgcccaagtatggctatgcgtcccccggagctctctacgcaacgcaaatgtatttcgagctggaaggtgtaggcggcctcaagccaggatactattactatcaacccgtacggcatcagttggtgctcatcagcgaacgcgaggccaccggtaaggctactgcacagatccactttattggtaagaaatccggtatcgaaccggtttataaaaataacatcttggaagttctggagattgaaaccggacatatggtcggtttgttcgagcaaattctgcccgcctacggcctggacattcacgaccgtgcgtacgagcccgcggttaaggatctgctcgacgttgcagacgaggactactatctgggcaccttcgagcttgttccccatgccggtgctcgtgatgaccaagcggaagtttatgttcagacccacggaggtaaggtggctggtctccctgagggacagtaccgctacgagaatggcgaattgactcggttttcagacgatatcgttctcaagaaacacgtaattgcgattaaccagagcgtctatcaggcagcgtcattcggcattagcgtatattctcgtgctgaggaagagtggttgaaatacattaccttgggaaaaaagcttcagcatctcatgatgaatggcctgaatttgggcttcatgagctctggttatagctccaagacgggtaatccgcttcctgcatcccgccgcatggatgccgtcctgggtgccaacggtgttgattcggctccaatgtacttttttgttggtggccgcatctccgacgagcagattggacacgagggcatgcgtgaggactcagttcacatgcggggtccagccgagctgatccgtgacgatcttgtgtcttttctgcccgactatatgatccctaatcgcgtagtagtctttgaccgccttccgctcagcgcgaatggtaaaattgatgttaaagcccttgcggcgtcagaccaggttaacgcagagttggtagaacgcccgttcgtagcccctcgcactgaaactgaaaaagaaattgctgcggtttgggaaaaagccctgcgccgcgaaaatgcttctgtacaggacgacttttttgagtccggaggcaactctctcatcgctgtcggtttggtccgtgaactcaacgctcgtctgggagtatctctgccgctgcaatcggtcttggagtcgccaacgatcgaaaagcttgcacgccgtctcgagcgggaggtggctcaagaatcatcccgcttcgtccggcttcatgccgagactggtaaggcccgtccggttatttgctggccgggcttgggcggttatcctatgaacctccgttcactggccggagagatcggacttggccgttcattctatggagttcagtcatacggcatcaacgagggtgagactccatacgagacgatcacggaaatggcaaaaaaggacattgaggcattgaaggaaattcaacccgcaggtccctacacgttgtggggctactcattcggagctcgggtcgcttttgagacggcttaccaattggaacaagccggtgaaaaggtagacaatttgtttctgatcgcccctggttcgcctaaggtgcgcgcggagaacggcaaagtttggggtcgggaggcgtcttttgccaatcgcggctatacgaccatccttttctcggtttttactggcactatttcgggaccggacctcgaccgctgcctggagaccgtaaccgatgaggcctcgttcgccgaatttatcagcgagttgaagggaattgatgtggaccttgcacggcggattatttcagtggtgggacaaacgtacgagttcgagtattcctttcatgagcttgcagaacgtactcttcaggcacccatttccatctttaaagccgtgggtgacgactactcattcctcgagaattcttcgggttattcggctgaaccacctacggtgatcgatctcgatgccgatcactactcgcttctccgtgaggatatcggcgaacttgtaaagcatattcgttaccttctcggtgagtaagaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccatgctggatgagtctttgtttccaaattcggcaaagttttctttcattaaaactggcgatgctgttaatttagaccatttccatcagttgcatccgttggaaaaggcactggtagcgcactcggttgatattagaaaagcagagtttggagatgccaggtggtgtgcacatcaggcactccaagctttgggacgagatagcggtgatcccattttgcgtggggaacgaggaatgccattgtggccttcttcggtgtctggttcattgacccacactgacggattccgagctgctgttgtggcgccacgattgttggtgcgttctatgggattggatgccgaacctgcggagccgttgcccaaggatgttttgggttcaatcgctcgggtgggggagattcctcaacttaagcgcttggaggaacaaggtgtgcactgcgcggatcgcctgctgttttgtgccaaggaagcaacatacaaagcgtggttcccgctgacgcataggtggcttggttttgaacaagctgagatcgacttgcgtgatgatggcacttttgtgtcctatttgctggttcgaccaactccagtgccgtttatttcaggtaaatgggtactgcgtgatggttatgtcatagctgcgactgcagtgacttgagcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 32 - DNA, 4517 bp, synthetic construct
  source 1..4517: mol_type = other DNA
catagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttctagaactagtatgactttgcaagaaacttctgttttggaaccaactttgcaaggtactactactttgccaggtttgttggctcaaagagttgctgaacacccagaagctatcgctgttgcttacagagacgacaagttgactttcagagaattggcttctagatctgctgctttggctgactacttggaacacttgggtgtttctgctgacgactgtgttggtttgttcgttgaaccatctatcgacttgatggttggtgcttggggtatcttgaacgctggtgctgcttacttgccattgtctccagaatacccagaagacagattgagatacatgatcgaaaactctgaaactaagatcatcttggctcaacaaagattggtttctagattgagagaattggctccaaaggacgttactatcgttactttgagagaatctgaagctttcgttagaccagaaggtactgaagctccagctgctagatctgctagaccagacactttggcttacgttatctacacttctggttctactggtaagccaaagggtgttatgatcgaacacagatctatcgttaaccaattgggttggttgagagaaacttacgctatcgacagatctaaggttatcttgcaaaagactccaatgtctttcgacgctgctcaatgggaaatcttgtctccagctaacggtgctactgttgttatgggtgctccaggtgtttacgctgacccagaaggtttgatcgaaactatcgttaagcacaacgttactactttgcaatgtgttccaactttgttgcaaggtttgatcgacactgaaaagttcccagaatgtgtttctttgcaacaaatcttctctggtggtgaagctttgtctagattgttggctatccaaactactcaagaaatgccaggtagagctttgatcaacgtttacggtccaactgaaactactatcaactcttcttctttcccagttgacccagctgacttggacgaaggtccacaatctatctctatcggttctccagttcacggtactacttaccacatcttggacaaggaaactttgaagccagttggtgttggtgaaatcggtgaattgtacatcggtggtatccaattggctagaggttacttgcacagagacgacttgactgctgaaagattcttggaaatcgaattggaagaaggtgctgaaccagttagattgtacaagactggtgacttgggtcaatggaacaacgacggtactgttcaattcgctggtagagctgacaaccaagttaagttgagaggttacagagttgaattggacgaaatctctttggctatcgaaaaccacgactgggttagaaacgctgctgttatcgttaagaacgacggtagaactggtttccaaaacttgatcgcttgtatcgaattgtctgaaaaggaagctgctttgatggaccaaggtaaccacggttctcaccacgcttctaagaagtctaagttgcaagttaaggctcaattgtctaacccaggtttgagagacgacgctgaattggctgctagaccagctttcgacttggaaggtgctgaaccaactccagaacaaagagctagagttttcgctagaaagacttacagattctacgaaggtggtgctgttactcaagctgacttgttgggtttgttgggtgctactgttactgctggttactctagaaaggctgctgacttggctccagctgaattgggtcaaatcttgagatggttcggtcaatacatctctgaagaaagattgttgccaaagtacggttacgcttctccaggtgctttgtacgctactcaaatgtacttcgaattggaaggtgttggtggtttgaagccaggttactactactaccaaccagttagacaccaattggttttgatctctgaaagagaagctactggtaaggctactgctcaaatccacttcatcggtaagaagtctggtatcgaaccagtttacaagaacaacatcttggaagttttggaaatcgaaactggtcacatggttggtttgttcgaacaaatcttgccagcttacggtttggacatccacgacagagcttacgaaccagctgttaaggacttgttggacgttgctgacgaagactactacttgggtactttcgaattggttccacacgctggtgctagagacgaccaagctgaagtttacgttcaaactcacggtggtaaggttgctggtttgccagaaggtcaatacagatacgaaaacggtgaattgactagattctctgacgacatcgttttgaagaagcacgttatcgctatcaaccaatctgtttaccaagctgcttctttcggtatctctgtttactctagagctgaagaagaatggttgaagtacatcactttgggtaagaagttgcaacacttgatgatgaacggtttgaacttgggtttcatgtcttctggttactcttctaagactggtaacccattgccagcttctagaagaatggacgctgttttgggtgctaacggtgttgactctgctccaatgtacttcttcgttggtggtagaatctctgacgaacaaatcggtcacgaaggtatgagagaagactctgttcacatgagaggtccagctgaattgatcagagacgacttggtttctttcttgccagactacatgatcccaaacagagttgttgttttcgacagattgccattgtctgctaacggtaagatcgacgttaaggctttggctgcttctgaccaagttaacgctgaattggttgaaagaccattcgttgctccaagaactgaaactgaaaaggaaatcgctgctgtttgggaaaaggctttgagaagagaaaacgcttctgttcaagacgacttcttcgaatctggtggtaactctttgatcgctgttggtttggttagagaattgaacgctagattgggtgtttctttgccattgcaatctgttttggaatctccaactatcgaaaagttggctagaagattggaaagagaagttgctcaagaatcttctagattcgttagattgcacgctgaaactggtaaggctagaccagttatctgttggccaggtttgggtggttacccaatgaacttgagatctttggctggtgaaatcggtttgggtagatctttctacggtgttcaatcttacggtatcaacgaaggtgaaactccatacgaaactatcactgaaatggctaagaaggacatcgaagctttgaaggaaatccaaccagctggtccatacactttgtggggttactctttcggtgctagagttgctttcgaaactgcttaccaattggaacaagctggtgaaaaggttgacaacttgttcttgatcgctccaggttctccaaaggttagagctgaaaacggtaaggtttggggtagagaagcttctttcgctaacagaggttacactactatcttgttctctgttttcactggtactatctctggtccagacttggacagatgtttggaaactgttactgacgaagcttctttcgctgaattcatctctgaattgaagggtatcgacgttgacttggctagaagaatcatctctgttgttggtcaaacttacgaattcgaatactctttccacgaattggctgaaagaactttgcaagctccaatctctatcttcaaggctgttggtgacgactactctttcttggaaaactcttctggttactctgctgaaccaccaactgttatcgacttggacgctgaccactactctttgttgagagaagacatcggtgaattggttaagcacatcagatacttgttgggtgaataactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgc

SEQ ID NO: 33 - DNA, 1384 bp, synthetic construct
  source 1..1384: mol_type = other DNA
cacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaagcggccgcatgttggacgaatctttgttcccaaactctgctaagttctctttcatcaagactggtgacgctgttaacttggaccacttccaccaattgcacccattggaaaaggctttggttgctcactctgttgacatcagaaaggctgaattcggtgacgctagatggtgtgctcaccaagctttgcaagctttgggtagagactctggtgacccaatcttgagaggtgaaagaggtatgccattgtggccatcttctgtttctggttctttgactcacactgacggtttcagagctgctgttgttgctccaagattgttggttagatctatgggtttggacgctgaaccagctgaaccattgccaaaggacgttttgggttctatcgctagagttggtgaaatcccacaattgaagagattggaagaacaaggtgttcactgtgctgacagattgttgttctgtgctaaggaagctacttacaaggcttggttcccattgactcacagatggttgggtttcgaacaagctgaaatcgacttgagagacgacggtactttcgtttcttacttgttggttagaccaactccagttccattcatctctggtaagtgggttttgagagacggttacgttatcgctgctactgctgttacttaattaattaacaattcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctc

SEQ ID NO: 34 - DNA, 1029 bp, synthetic construct
  source 1..1029: mol_type = other DNA
atgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataa

SEQ ID NO: 35 - DNA, 4847 bp, synthetic construct
  source 1..4847: mol_type = other DNA
tgttcacattcgaacggtctctgctttgacaacatgctgtgcggtgttgtaaagtcgtggccaggagaatacgacagcgtgcaggactgggggagttggatccatgaccctccaggagacctcggtactggagcccaccctccaggggaccacgaccctcccaggcctgctggcccagcgcgtggcggagcacccggaggcgattgccgtggcgtaccgggacgacaagctcaccttccgggaactggcgtcccggtccgccgccctggccgactacctggagcatcttggtgtcagcgcggacgactgcgtgggcctgttcgtggagccgagcatcgacctcatggtgggagcctggggcatcctgaacgccggtgcggcctacctcccgctatcgccggagtacccagaggaccggctgcgctacatgatcgagaacagcgagaccaagatcatcctcgcccagcagcggctggtctcccgcctgcgggagctcgcccccaaggacgtcaccatcgtcaccctgcgggagagtgaggccttcgtccgccccgagggaaccgaggcaccggcggcccgctcggcgcgccccgacacgctggcctacgtgatctacacgtccggctcgacgggcaagccgaagggcgtcatgatcgagcaccggtcgatcgtcaaccagctcgggtggttgcgcgagacctacgctatcgaccgttccaaggtgatcctgcaaaagacgccgatgtcgttcgacgccgctcagtgggagatcctatccccggcgaacggtgccacggtggtgatgggcgccccgggcgtctacgccgaccccgagggcctgatcgaaaccatcgtcaagcacaacgtcacaaccctgcagtgcgtccccaccctgctccagggcctcatcgacaccgagaagttcccggagtgcgtgagcctccagcagatcttctcgggcggggaggccctgagccgactgctggccatccagaccacccaggagatgcccggaagggccctgatcaacgtctacggccctaccgaaaccacgatcaacagttcctcgttccccgtcgacccggccgacctcgacgagggcccccagtccatcagcatcgggtcgcccgtgcacggcaccacctaccacatcctggacaaggagacgctcaagcccgtgggggtcggcgagatcggcgagctgtacatcggcggcatccagctggcgcggggctatctgcaccgggacgacctgaccgcggagcgcttcctggagatcgagctggaggaaggggccgagccggtgcgcctgtacaagacgggcgacctcgggcagtggaacaacgacggcaccgtgcagttcgccgggcgggccgacaaccaggtcaagttgcgcggctaccgtgtggagctagacgaaatcagccttgccatcgagaaccatgactgggtccggaacgcagcggtcatcgtgaaaaacgacggccgcactggattccagaacctgatcgcctgcatcgaactctccgagaaggaggcggcgctgatggaccagggcaaccacggctcccaccacgcgagcaagaagtcaaagctccaggtgaaggcgcagctctctaaccccggcctccgtgacgacgcggaattggcggcgcgccccgccttcgacctcgagggtgcagaaccgacgcccgagcagcgggcgcgggtcttcgcccgcaagacctaccgcttctacgagggcggcgccgtcacccaggcggacctgctgggactcctgggcgccaccgtgacggccggatactcccgcaaagcggcggacctcgcacccgcggagctgggccagatcctgcgctggttcggccagtacattagcgaggagcgcctgctgccgaagtacgggtacgcctcccccggcgcgctctacgcgacgcagatgtacttcgagctggaaggggtaggcggcctcaagccgggctactactactaccagccggtgcggcaccagctggtgctgatcagcgagagagaggcgaccggaaaggccaccgcgcagatccacttcatcgggaagaagagcggcatcgagcccgtctacaagaacaacatcctggaggtcctggaaatcgagacaggccacatggtcggcctcttcgagcagatcctgcccgcctacggcctggacatccacgaccgggcctacgagccggcggtcaaggacctgctcgacgtagccgacgaggactactacctcgggacgttcgaactcgtcccgcacgccggcgcccgcgatgaccaggcggaggtctacgtccagacccacggcggcaaggtcgccggcctcccggagggccagtaccgctacgagaacggcgagctgacccgcttctccgacgacatcgtcctgaagaagcacgttatcgcgatcaaccagagcgtgtaccaggccgcgtcattcggcatctccgtgtacagccgggcagaggaggagtggctgaagtacatcaccctcggcaagaagctccagcacctgatgatgaacgggctgaacctaggcttcatgtcctcgggctacagctccaagaccggcaacccgctgcccgcctcgcgccggatggacgccgtactgggggccaatggggtcgacagcgcccccatgtacttcttcgtcggaggccgcatctcggacgagcagatcggccacgagggcatgcgggaggactccgtccacatgcgcggaccggccgaactgatccgcgacgacctggtctccttcctccccgactacatgatcccgaaccgggtggtcgtcttcgaccggctccccctgtcggcgaacggcaagatcgatgtgaaggcgcttgcggcctccgaccaagtgaacgccgagctggtggagcggccgttcgtggcgccgcgcaccgagaccgagaaggagatcgccgccgtgtgggagaaggcgctgcgccgcgagaacgcgtccgtccaggacgacttcttcgagtcggggggcaactccctcatcgccgtgggcctagtcagggagctcaacgcccggctgggtgtcagcctcccgctgcagtcggttctggagagccccacgatcgagaagctggcgaggaggctggagcgcgaggtggcccaggagagtagtcggttcgtgcgcctgcacgccgaaaccggcaaagcaagaccggtcatctgctggccgggtctaggcggttaccccatgaacttacggtccctcgcgggcgagatcggcctgggtcgctcgttctacggcgtgcagtcctacggtatcaacgagggggagacgccctacgagaccataacggagatggccaagaaggacatcgaggccctgaaggagatccagcctgcgggtccgtacaccctgtgggggtactccttcggggcccgggtcgccttcgaaaccgcctaccagctcgaacaggccggtgagaaggtggacaacctgtttctcatcgccccgggctcccccaaggtgcgggcggagaacggcaaggtgtggggccgggaggcatcattcgccaaccgcggctacaccaccatcctcttcagcgtgttcaccggcacaatctctgggcccgacctcgaccgctgtctggagaccgtcaccgacgaggcctccttcgcggagttcatcagcgagctgaagggcatcgacgtcgacctagcccgacgcatcatctccgtcgtcggccagacgtacgagttcgagtattccttccacgagctggccgagcgcacgctgcaagccccgatctccatcttcaaggccgtcggcgacgactactcgttcctggagaactcctccggctactccgccgaaccgccgacggtcatcgacctcgacgccgaccactactccctgctacgtgaagacatcggcgagctcgtcaagcacatccggtacctgctcggagaataaggatccactctgtcagatctcactctgccaaggaggacgcacatgctggacgagtccctgttcccgaacagcgcgaagttctccttcatcaagaccggcgacgccgtcaacctcgaccacttccaccagctgcacccgctggagaaggcgctggtcgcccactccgtcgacatccgcaaggccgagttcggcgacgcccggtggtgcgcgcatcaggccctccaggcgctcggtagggacagcggggacccgatcctccggggagaacgcggcatgccgctgtggccgtcctcggtgagcggctcgctgacccacaccgacgggttccgggccgccgtggtcgcgccccggctgctcgtgcgctcgatgggcctggacgcagagccggccgaacccctgcccaaggacgtgctggggtcgatcgcccgcgtcggcgagatcccgcagctcaagcgcctggaggagcagggcgtgcactgcgccgaccggctcctcttctgcgccaaggaggcgacgtacaaggcctggttccccctcacgcaccgctggctgggcttcgagcaggccgagatcgacctgcgcgacgacggcaccttcgtctcctacctgctggtgcggcccaccccggtgcccttcatctccggcaagtgggtcctccgtgatggctacgtcatcgcggctaccgcggtcacgtaagaattcagatctacgcgttccccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttt

(19) United States
(12) Patent Application Publication
(10) Pub. No.: US 2025/0354185 A1
(43) Pub. Date: Nov. 20, 2025

(54) BLUE PIGMENT AND BIOSYNTHESIS METHOD THEREOF

(71) Applicant: VERTEXYN BIOWORKS CO., LTD., Nanjing (CN)

(72) Inventors: Lei Sun, Nanjing (CN); Fuchao Xu, Nanjing (CN); Xiaoxiao Song, Nanjing (CN); Jingjing Bi, Nanjing (CN)

(21) Appl. No.: 19/241,420

(22) Filed: Jun. 18, 2025

Related U.S. Application Data
(63) Continuation of application No. PCT/CN2024/095947, filed on May 29, 2024.

(30) Foreign Application Priority Data
May 14, 2024 (CN) 202410592631.0

(51) Int. Cl. C12P 17/16; C07D 403/04; C09B 53/02; C12R 1/15; C12R 1/19; C12R 1/465; C12R 1/865

(52) U.S. Cl. CPC C12P 17/165; C07D 403/04; C09B 53/02; C12R 2001/15; C12R 2001/19; C12R 2001/465; C12R 2001/865

(57) ABSTRACT

The present application relates to the technical field of biocatalysis and biosynthesis, and specifically discloses a blue pigment and a biosynthesis method thereof. In the present application, an indigoidine synthetase and a 4′-phosphopantetheinyl transferase are expressed by a metabolically engineered strain to catalyze the biosynthesis of the blue pigment N-acetyl-indigoidine from glutamine and N-acetylglutamine, and a molecular structure of the blue pigment is inferred by mass spectrometry, nuclear magnetic resonance spectroscopy, etc. The present application achieves the catalytic synthesis of N-acetyl-indigoidine from glutamine and N-acetylglutamine in Escherichia coli (E. coli), Corynebacterium glutamicum (C. glutamicum), Saccharomyces cerevisiae (S. cerevisiae), and Streptomyces. Compared with indigoidine, N-acetyl-indigoidine has a maximum absorption wavelength of 584 nm, and a stable color having high brightness that is not easy to fade. Thus, the blue pigment shows an extensive application range and a promising industrial production prospect.

### CROSS-REFERENCE TO RELATED APPLICATIONS

The present application is a Continuation Application of PCT application No. PCT/CN2024/095947 filed on May 29, 2024, which claims the benefit of Chinese Patent Application No. 202410592631.0 filed on May 14, 2024. The contents of the above-identified applications are hereby incorporated by reference.

### REFERENCE TO SEQUENCE LISTING

This application includes a Sequence Listing filed electronically as an XML file named “US2501029H-PCT_SL.xml”, created on Jun. 15, 2025, with a size of 62,396 bytes. The Sequence Listing is incorporated herein by reference.

### TECHNICAL FIELD

The present application relates to the technical field of biocatalysis and biosynthesis, and specifically relates to a blue pigment and a biosynthesis method thereof.

### BACKGROUND

Indigoidine is a blue, non-toxic natural product of bacteria, which is synthesized through the condensation of two L-glutamine molecules under the catalysis of indigoidine synthetase, a non-ribosomal peptide synthetase (NRPS). Indigoidine has a bright and deep blue color comparable to the color of indigo, and can be used as a novel natural blue pigment and dye in the printing and dyeing, food, and pharmaceutical industries. Indigoidine has been reported to be used for the dyeing of proteinaceous fiber textiles.

The Chinese patent CN109722401A discloses the co-expression of a coding gene (bpsA) for an indigoidine synthetase from Streptomyces lavendulae (S. lavendulae) and a coding gene (sfp) for a 4′-phosphopantetheinyl transferase from Bacillus subtilis (B. subtilis) in Corynebacterium glutamicum (C. glutamicum). In this patent, when L-glutamine as a substrate is added at an amount of 11.68 g/L and the induction culture is conducted with 0.8 mM isopropylthio-ß-galactoside (IPTG) at 18° C. for 48 h, a yield of indigoidine reaches 1.75 g/L. The Chinese patent CN109722401A discloses the co-expression of coding genes for the indigoidine synthetase, 4′-phosphopantetheinyl transferase, and glutamine synthetase in Escherichia coli (E. coli). In this patent, with L-glutamate as a substrate, the bioconversion is conducted to prepare indigoidine. After the bioconversion is conducted for 24 h, a yield of indigoidine is 5.38 g/L. Wehrs, M. et al. integrate a coding gene (bpsA) for the indigoidine synthetase from S. lavendulae and a coding gene (sfp) for the 4′-phosphopantetheinyl transferase from B. subtilis into a genome of Saccharomyces cerevisiae (S. cerevisiae). After a resulting strain is cultured in a 2 L fermentation tank for 72 h with glucose as a substrate, a yield of indigoidine reaches 980 mg/L (Production efficiency of the bacterial non-ribosomal peptide indigoidine relies on the respiratory metabolic state in S. cerevisiae. Microb. Cell Fact. 2018, 17, No. 193). Ming Zhao et al. establish an efficient expression regulation system in Streptomyces lividans (S. lividans) to accurately regulate a coding gene (indC) for the indigoidine synthetase derived from Streptomyces albus J1704. Accordingly, after the culture is allowed in a 4 L fermentation tank for 72 h with glycerol as a substrate, an output of indigoidine reaches 46.27 g/L (Establishment of an Efficient Expression and Regulation System in Streptomyces for Economical and High-Level Production of the Natural Blue Pigment Indigoidine. J Agric Food Chem Journal of Agricultural and Food Chemistry 2024 72 (1), 483-492).

The above studies achieve the biosynthesis of indigoidine in C. glutamicum, E. coli, S. cerevisiae, and S. lividans with the indigoidine synthetase from different sources and different substrates. However, none of these studies involves N-acetyl-indigoidine.

### SUMMARY

An objective of the present application is to overcome the deficiencies of the prior art and provide a blue pigment and a biosynthesis method thereof. Due to a protective effect of acetyl, the blue pigment (N-acetyl-indigoidine) obtained in the present application exhibits lower polarity and water solubility than indigoidine, is not easy to fade, and has a bright color. The blue pigment shows an extensive application range and a promising industrial production prospect.

To achieve the above objective, the present application adopts the following technical solutions:

The present application provides a blue pigment having a chemical name of N-acetyl-indigoidine, a molecular formula of C₁₂H₁₀N₄O₅, and a chemical structure set forth in the following formula I:

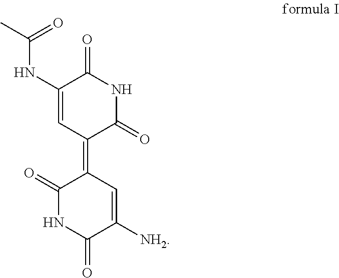 [SMILES: CC(=O)NC1=C/C(=C2/C=C(N)C(=O)NC2=O)C(=O)NC1=O]

In the present application, a novel blue pigment (N-acetyl-indigoidine) is obtained. It is determined by mass spectrometry and nuclear magnetic resonance spectroscopy that the blue pigment has a molecular structure set forth in the formula I, a molecular formula of C₁₂H₁₀N₄O₅, a relative molecular mass of 289.9, a maximum absorption wavelength of 584 nm, and characteristic peaks of —C═O and —CH₃ in acetyl at δ=172.23 ppm and δ=24.90 ppm on a ¹³C CPMAS nuclear magnetic resonance spectrum. N-acetyl-indigoidine has a more vibrant and brighter color than indigoidine.

Amino and acetyl in N-acetyl-indigoidine form an amide structure, which reduces the polarity and hydrophilicity of N-acetyl-indigoidine. In contrast to indigoidine, N-acetyl-indigoidine is stable, is not easy to fade, and shows excellent color brightness. N-acetyl-indigoidine has an extensive application range and a promising industrial production prospect.

The present application also provides a biosynthesis method of the blue pigment, including: expressing an indigoidine synthetase and a 4′-phosphopantetheinyl transferase by a metabolically engineered strain to catalyze biosynthesis of the blue pigment from glutamine and N-acetylglutamine.

Through a large number of experiments, the present application has found that the indigoidine synthetase and 4′-phosphopantetheinyl transferase expressed by the metabolically engineered strain can catalyze the biosynthesis of the blue pigment (N-acetyl-indigoidine) with a mixture of glutamine and N-acetylglutamine as a substrate, while the metabolically engineered strain cannot synthesize the N-acetyl-indigoidine with the glutamine or N-acetylglutamine alone as a substrate.

As a preferred embodiment of the biosynthesis method of the blue pigment in the present application, a coding gene for the indigoidine synthetase includes a coding gene bpsA for the indigoidine synthetase or a coding gene indC for the indigoidine synthetase; and
a coding gene for the 4′-phosphopantetheinyl transferase includes a coding gene EntD for the 4′-phosphopantetheinyl transferase or a coding gene indB for the 4′-phosphopantetheinyl transferase.

The present application has found for the first time that a metabolically engineered strain carrying the coding gene bpsA for the indigoidine synthetase and the coding gene EntD for the 4′-phosphopantetheinyl transferase can synthesize the novel compound N-acetyl-indigoidine with a mixture of glutamine and N-acetylglutamine as a substrate. Experiments show that the metabolically engineered strain cannot synthesize the N-acetyl-indigoidine with the glutamine or N-acetylglutamine alone as a substrate.

In the present application, the introduction of the coding gene indC for the indigoidine synthetase and the coding gene indB for the 4′-phosphopantetheinyl transferase into a host strain can also allow the synthesis of the novel compound N-acetyl-indigoidine with a mixture of glutamine and N-acetylglutamine as a substrate.

In some specific embodiments, the coding gene bpsA for the indigoidine synthetase has a nucleotide sequence set forth in SEQ ID NO: 1, the coding gene indC for the indigoidine synthetase has a nucleotide sequence set forth in SEQ ID NO: 2, the coding gene EntD for the 4′-phosphopantetheinyl transferase has a nucleotide sequence set forth in SEQ ID NO: 3, and the coding gene indB for the 4′-phosphopantetheinyl transferase has a nucleotide sequence set forth in SEQ ID NO: 4.

The indigoidine synthetase and the 4′-phosphopantetheinyl transferase involved in the present application can be derived from any species. For example, the indigoidine synthetase is derived from S. lavendulae, and a coding gene bpsA for the indigoidine synthetase has a nucleotide sequence set forth in SEQ ID NO: 1. Alternatively, the indigoidine synthetase is derived from Streptomyces chromofuscus (S. chromofuscus) ATCC49982, and a coding gene indC for the indigoidine synthetase has a nucleotide sequence set forth in SEQ ID NO: 2. For example, the 4′-phosphopantetheinyl transferase is derived from C. glutamicum, and a coding gene entD for the 4′-phosphopantetheinyl transferase has a nucleotide sequence set forth in SEQ ID NO: 3. Alternatively, the 4′-phosphopantetheinyl transferase is derived from S. chromofuscus ATCC49982, and a coding gene indC for the 4′-phosphopantetheinyl transferase has a nucleotide sequence set forth in SEQ ID NO: 4. The above are merely examples, and other sources of the indigoidine synthetase and the 4′-phosphopantetheinyl transferase also fall within the protection scope of the present application.

As a preferred embodiment of the biosynthesis method of the blue pigment in the present application, a construction process of the metabolically engineered strain includes:
introducing a coding gene bpsA for the indigoidine synthetase and a coding gene EntD for the 4′-phosphopantetheinyl transferase into a host strain to construct the metabolically engineered strain; or
introducing a coding gene indC for the indigoidine synthetase- and a coding gene indB for the 4′-phosphopantetheinyl transferase into the host strain to construct the metabolically engineered strain.

As a preferred embodiment of the biosynthesis method of the blue pigment in the present application, the construction process of the metabolically engineered strain includes the following steps:

amplifying the coding gene bpsA for the indigoidine synthetase and the coding gene EntD for the 4′-phosphopantetheinyl transferase separately through polymerase chain reaction (PCR), and ligating amplified fragments to a plasmid to produce the metabolically engineered strain; or

amplifying the coding gene indC for the indigoidine synthetase and the coding gene indB for the 4′-phosphopantetheinyl transferase separately through PCR, and ligating amplified fragments to the plasmid to produce the metabolically engineered strain.

In some specific embodiments, the metabolically engineered strain is a recombinant E. coli strain HG-N-Idg01, and the recombinant E. coli strain HG-N-Idg01 is constructed by introducing an expression vector pCDFDuet-bpsA-entD into E. coli BL21 (DE3).

The expression vector pCDFDuet-bpsA-entD is produced through PCR amplification of the coding gene bpsA for the indigoidine synthetase derived from S. lavendulae and the coding gene EntD for the 4′-phosphopantetheinyl transferase derived from C. glutamicum, gene synthesis, and ligation to an expression vector pCDFDuet-1.

Preferably, a construction process of the recombinant E. coli strain HG-N-Idg-01 includes the following specific steps:
(1) synthesizing the coding gene bpsA for the indigoidine synthetase and the coding gene entD for the 4′-phosphopantetheinyl transferase by Tsingke Biotechnology Co., Ltd.;
(2) inserting bpsA and entD gene fragments into two multiple cloning sites of a plasmid pCDFDuet-1 with a seamless cloning kit of Takara Bio respectively to produce a recombinant plasmid pCDFDuct-bpsA-entD; and
(3) transforming the recombinant plasmid pCDFDuet-bpsA-entD into E. coli BL21 (DE3) through chemical transformation to produce the recombinant E. coli strain HG-N-Idg-01.

In some specific embodiments, the metabolically engineered strain is a recombinant E. coli strain HG-N-Idg02 overexpressing a coding gene (indC) for the indigoidine synthetase derived from S. chromofuscus ATCC49982 and a coding gene (indB) for the 4′-phosphopantetheinyl transferase, and the recombinant E. coli strain HG-N-Idg02 is constructed by introducing an expression vector pCDFDuet-indC-indB into E. coli BL21 (DE3).

The expression vector pCDFDuet-bpsA-entD is produced through PCR amplification of the coding gene indC for the indigoidine synthetase and the coding gene indB for the 4′-phosphopantetheinyl transferase, gene synthesis, and ligation to an expression vector pCDFDuet-1.

In some specific embodiments, the metabolically engineered strain is one selected from the group consisting of a recombinant C. glutamicum strain HG-N-Idg03, a recombinant S. cerevisiae strain HG-N-Idg04, and a recombinant S. lividans strain HG-N-Idg05.

The recombinant C. glutamicum strain HG-N-Idg03 is constructed by introducing an expression vector pXMJ19-bpsA-entD into C. glutamicum ATCC13032.

The expression vector pXMJ19-bpsA-entD is produced through PCR amplification of the coding gene bpsA for the indigoidine synthetase and the coding gene EntD for the 4′-phosphopantetheinyl transferase and ligation to an expression vector pXMJ19.

Preferably, a construction process of the recombinant C. glutamicum strain HG-N-Idg03 includes the following specific steps:
(1) acquiring the coding gene bpsA for the indigoidine synthetase and the coding gene EntD for the 4′-phosphopantetheinyl transferase through PCR amplification with a plasmid pCDF-bpsA-entD as a template;
(2) inserting bpsA and entD gene fragments into multiple cloning sites of a plasmid pXMJ19 with a seamless cloning kit of Takara Bio to produce a recombinant plasmid pXMJ19-bpsA-entD; and
(3) transforming the recombinant plasmid pXMJ19-bpsA-entD into C. glutamicum ATCC13032 through electroporation to produce the recombinant C. glutamicum strain HG-N-Idg03.

The recombinant S. cerevisiae strain HG-N-Idg04 is constructed by introducing an expression vector pRS425-bpsA-entD into S. cerevisiae INVSC1.

The expression vector pRS425-bpsA-entD is produced through PCR amplification of the coding gene bpsA for the indigoidine synthetase derived from S. lavendulae and the coding gene EntD for the 4′-phosphopantetheinyl transferase derived from C. glutamicum, gene synthesis, and ligation to an expression vector pRS425.

Preferably, a construction process of the recombinant strain HG-N-Idg-04 includes the following specific steps:
(1) synthesizing the coding gene bpsA for the indigoidine synthetase and the coding gene EntD for the 4′-phosphopantetheinyl transferase by Tsingke Biotechnology Co., Ltd.;
(2) inserting bpsA and entD gene fragments into multiple cloning sites of a plasmid pRS425 to produce a recombinant plasmid pRS425-bpsA-entD; and
(3) transforming the recombinant plasmid pRS425-bpsA-entD into S. cerevisiae INVSC1 through lithium acetate transformation to produce the recombinant strain HG-N-Idg-04.

The recombinant S. lividans strain HG-N-Idg05 is constructed by introducing an expression vector pKCH-PkasO-bpsA-entD into S. lividans TK24.

The expression vector pKCH-PkasO-bpsA-entD is produced through PCR amplification of a hygromycin B resistance gene (hyg^{r}, a PkasO promoter, the coding gene bpsA for the indigoidine synthetase derived from S. lavendulae, and the coding gene EntD for the 4′-phosphopantetheinyl transferase derived from C. glutamicum, gene synthesis, and ligation to a plasmid pKC1139.

Preferably, a construction process of the recombinant S. lividans strain HG-N-Idg05 includes the following specific steps:
(1) synthesizing a sequence of the hygromycin B resistance gene and a sequence of the PkasO-bpsA-entD gene by Tsingke Biotechnology Co., Ltd.;
(2) inserting the sequence of the hygromycin B resistance gene and the sequence of the PkasO-bpsA-entD gene into multiple cloning sites of a plasmid pKC1139 to produce a recombinant plasmid pKCH-PkasO-bpsA-entD; and
(3) transforming the recombinant plasmid pKCH-PkasO-bpsA-entD into S. lividans TK24 through conjugative transfer to produce the recombinant S. lividans strain HG-N-Idg05.

The recombinant E. coli strains HG-N-Idg01 and HG-N-Idg02, the recombinant C. glutamicum strain HG-N-Idg03, the recombinant S. cerevisiae strain HG-N-Idg04, and the recombinant S. lividans strain HG-N-Idg05 constructed in the present application efficiently express the indigoidine synthetase and 4′-phosphopantetheinyl transferase through plasmids, and all can catalyze the biosynthesis of N-acetyl-indigoidine from glutamine and N-acetylglutamine as a substrate.

The metabolically engineered strain in the present application can be used as a cell catalyst to catalyze the synthesis of the novel compound N-acetyl-indigoidine from a substrate.

As a preferred embodiment of the biosynthesis method of the blue pigment in the present application, the plasmid includes at least one selected from the group consisting of pCDFDuct, pXMJ19, pRS425, and pKC1139 plasmids.

As a preferred embodiment of the biosynthesis method of the blue pigment in the present application, the PCR amplification is conducted using primers having sequences set forth in SEQ ID NOs: 5-30.

As a preferred embodiment of the biosynthesis method of the blue pigment in the present application, the host strain includes at least one selected from the group consisting of E. coli, C. glutamicum, S. cerevisiae, and S. lividans.

Preferably, the E. coli includes E. coli BL21 (DE3), the C. glutamicum includes C. glutamicum ATCC13032, the S. cerevisiae includes S. cerevisiae INVSC1, and the S. lividans includes S. lividans TK24.

The original E. coli BL21 (DE3), C. glutamicum ATCC13032, S. cerevisiae INVSC1, and S. lividans TK24 cannot synthesize N-acetyl-indigoidine with glutamine and N-acetylglutamine. The relevant enzyme genes need to be introduced into the E. coli, C. glutamicum, S. cerevisiae, and S. lividans to construct metabolically engineered strains only which can achieve the biocatalytic synthesis of N-acetyl-indigoidine.

As a preferred embodiment of the biosynthesis method of the blue pigment in the present application, the biosynthesis method includes:
collecting cells produced after an induction culture of the metabolically engineered strain through centrifugation;
resuspending the cells with a catalytic solution including phosphate buffered saline, glutamine, and N-acetylglutamine;
allowing catalysis to produce a conversion solution; and collecting the blue pigment in the conversion solution through centrifugation.

As a preferred embodiment of the biosynthesis method of the blue pigment in the present application, in the catalytic solution, a concentration of the glutamine is 0.1 g/L to 10 g/L and a concentration of the N-acetylglutamine is 0.1 g/L to 10 g/L.

Preferably, the concentration of the glutamine is 1 g/L and the concentration of the N-acetylglutamine is 1 g/L.

The present application also provides a purification method for the N-acetyl-indigoidine, including: collecting a solid precipitate in the conversion solution through centrifugation; adding the solid precipitate to N,N-dimethylformamide (DMF) for extraction of N-acetyl-indigoidine, and evaporating the DMF through vacuum lyophilization; washing a resulting solid with ultrapure water, methanol, ethyl acetate, and n-hexane successively; and conducting vacuum lyophilization once again to produce an N-acetyl-indigoidine sample.

The present application also provides a cell catalyst including the above-mentioned metabolically engineered strain.

In some specific embodiments, the present application provides a cell catalyst including the recombinant E. coli strain HG-N-Idg01, and a preparation process of the cell catalyst includes the following specific steps: picking a single colony of the recombinant E. coli strain HG-N-Idg01 and transferring the single colony to 5 mL of a Luria-Bertani (LB) medium including 50 μg/mL of streptomycin, and culturing at 37° C. and 220 rpm for 16 h; inoculating a resulting culture into 50 mL of a ZYM medium at an inoculum size of 1%, adding streptomycin at a final concentration of 50 μg/mL and an inducer IPTG at a final concentration of 0.1 mM, and culturing at 30° C. and 220 rpm for 24 h; and centrifuging to collect cells, which are the cell catalyst.

In some specific embodiments, the present application provides a cell catalyst including the recombinant C. glutamicum strain HG-N-Idg03, and a preparation process of the cell catalyst includes the following specific steps: picking a single colony of the recombinant C. glutamicumstrain HG-N-Idg03 and transferring the single colony to 5 mL of a BHISG medium including 15 μg/mL of chloramphenicol and 50 μg/L of biotin, and culturing at 30° C. and 220 rpm for 16 h; inoculating a resulting culture into 50 mL of a GAP medium at an inoculum size of 1%, adding chloramphenicol at a final concentration of 15 μg/mL and biotin at a final concentration of 50 μg/L, and culturing at 30° C. and 220 rpm for 3 h; then adding IPTG at a final concentration of 1 mM, and further culturing for 24 h; and centrifuging to collect cells, which are the cell catalyst.

In some specific embodiments, the present application provides a cell catalyst including the recombinant S. cerevisiae strain HG-N-Idg04, and a preparation process of the cell catalyst includes the following specific steps: picking a single colony of the recombinant S. cerevisiae strain HG-N-Idg04 and inoculating the single colony into 5 mL of an SC-Leu liquid medium, and culturing at 30° C. and 220 rpm for 24 h; centrifuging at 4° C. and 1,500 g for 5 min to produce a cell pellet and a supernatant, and removing the supernatant; resuspending the cell pellet with 30 mL of an SC-Leu liquid medium, adding 10 g/L of galactose, and culturing at 30° C. and 220 rpm for 24 h; and centrifuging to collect cells, which are the cell catalyst.

In some specific embodiments, the present application provides a cell catalyst including the recombinant S. lividans strain HG-N-Idg05, and a preparation process of the cell catalyst includes the following specific steps: picking the recombinant S. lividans strain HG-N-Idg05 grown on an MS medium for 4 d to 5 d by a sterile pipette tip, inoculating the recombinant S. lividans strain in a primary shake flask with 10 mL of a TSB medium, and culturing at 28° C. for 48 h; collecting 1 mL of a resulting culture and transferring it to a secondary shake flask with 50 mL of a TSB medium, and culturing at 28° C. for 72 h; and centrifuging to collect cells, which are the cell catalyst.

Compared with the prior art, the present application has the following beneficial effects:

The present application provides a blue pigment and a biosynthesis method thereof. Due to the protective effect of acetyl, the blue pigment (N-acetyl-indigoidine) obtained in the present application has a maximum absorption wavelength of 584 nm, reduced polarity and water solubility, and a bright color uneasy to fade compared with indigoidine. Thus, the blue pigment has an extensive application range and a promising industrial production prospect. In the present application, an indigoidine synthetase and a 4′-phosphopantetheinyl transferase are expressed by a metabolically engineered strain to catalyze the biosynthesis of N-acetyl-indigoidine from glutamine and N-acetylglutamine. A structure of N-acetyl-indigoidine is inferred, and it is identified that N-acetyl-indigoidine can be used as a novel blue pigment. The present application has also found that the catalytic synthesis of N-acetyl-indigoidine from glutamine and N-acetylglutamine can be achieved in strains such as E. coli, C. glutamicum, S. cerevisiae, and Streptomyces.

### BRIEF DESCRIPTION OF THE DRAWINGS

FIG. 1A to FIG. 1E show liquid chromatography spectra of products converted from different substrates under the catalysis of HG-N-Idg01;

FIG. 1A shows a liquid chromatography spectrum of an indigoidine standard;

FIG. 1B shows a liquid chromatography spectrum of a product converted from a mixture of glutamine and N-acetylglutamine under the catalysis of a strain BL21 (DE3);

FIG. 1C shows a liquid chromatography spectrum of a product converted from glutamine under the catalysis of the strain HG-N-Idg01;

FIG. 1D shows a liquid chromatography spectrum of a product converted from N-acetylglutamine under the catalysis of the strain HG-N-Idg01; and

FIG. 1E shows a liquid chromatography spectrum of a product converted from a mixture of glutamine and N-acetylglutamine under the catalysis of the strain HG-N-Idg01;

FIG. 2A shows an ultraviolet-visible absorption spectrum of indigoidine and FIG. 2B shows an ultraviolet-visible absorption spectrum of N-acetyl-indigoidine;

FIG. 3 shows a liquid chromatography-mass spectrometry (LC-MS) spectrum of N-acetyl-indigoidine;

FIG. 4 shows a proton nuclear magnetic resonance (¹H-NMR) spectrum of N-acetyl-indigoidine;

FIG. 5 shows a carbon-13 nuclear magnetic resonance (¹³C-NMR) spectrum of N-acetyl-indigoidine;

FIG. 6 shows a heteronuclear single quantum coherence (HSQC) NMR spectrum of N-acetyl-indigoidine;

FIG. 7 shows a heteronuclear multiple bond correlation (HMBC) NMR spectrum of N-acetyl-indigoidine;

FIG. 8 shows an inferred structure of N-acetyl-indigoidine;

FIG. 9 shows a cross-polarization magic angle spinning (CPMAS) NMR spectrum of indigoidine;

FIG. 10 shows a CPMAS NMR spectrum of N-acetyl-indigoidine;

FIG. 11 shows a biosynthetic route of N-acetyl-indigoidine by the strain HG-N-Idg01;

FIG. 12A shows a liquid chromatography spectrum of a catalytic product of HG-N-Idg01 and FIG. 12B shows a liquid chromatography spectrum of a catalytic product of HG-N-Idg02;

FIG. 13A shows a liquid chromatography spectrum of a catalytic product of C. glutamicum 13032 and FIG. 13B shows a liquid chromatography spectrum of a catalytic product of HG-N-Idg03;

FIG. 14A shows a liquid chromatography spectrum of a catalytic product of S. cerevisiae INVSc1 and FIG. 14B shows a liquid chromatography spectrum of a catalytic product of HG-N-Idg04;

FIG. 15A shows a liquid chromatography spectrum of a catalytic product of S. lividans TK24 and FIG. 15B shows a liquid chromatography spectrum of a catalytic product of HG-N-Idg05;

FIG. 16A and FIG. 16B show the comparison of colors of solutions of N-acetyl-indigoidine (FIG. 16A) and indigoidine (FIG. 16B) in dimethyl sulfoxide (DMSO); and

FIG. 17A and FIG. 17B show the comparison of colors of solutions of N-acetyl-indigoidine (FIG. 17A) and indigoidine (FIG. 17B) in water.

### DETAILED DESCRIPTION

To well explain the objective, technical solutions, and advantages of the present application, the present application will be further described below with reference to the accompanying drawings and specific embodiments.

In the following embodiments, unless otherwise specified, the experimental methods are conventional, and the materials, the reagents, etc. are commercially available.

The medium compositions involved in the following embodiments are as follows:

LB liquid medium: 10 g/L of peptone, 5 g/L of a yeast extract, and 10 g/L of NaCl. This liquid medium is sterilized at 121° C. for 20 min.

LB solid medium: 10 g/L of peptone, 5 g/L of the yeast extract, 10 g/L of NaCl, and 15 g/L of an agar powder. This solid medium is sterilized at 121° C. for 20 min and then cooled to about 50° C., a required antibiotic is added, and a resulting system is poured into plates, allowed to be solidified, and stored at 4° C. for later use.

ZYM fermentation medium: 96 mL of a ZY medium+2 mL of 50×M salts+2 mL of 50×5052+200 μL of 1 mol/L magnesium sulfate+100 μL of 1,000×trace elements, and 20 g/L of glucose.

ZY medium: 10 g/L of peptone and 5 g/L of the yeast extract. The ZY medium is sterilized at 121° C. for 20 min and stored for later use.

50×M salts: 1.25 mol/L of Na₂HPO₄, 1.25 mol/L of KH₂PO₄, 2.5 mol/L of NH₄Cl, and 0.25 mol/L of Na₂SO₄.

50×5052:250 g/L of glycerol, 25 g/L of glucose, and 1 mol/L of MgSO₄.

1,000×trace elements: 50 mmol/L of FeCl_{3,}20 mmol/L of CaCl₂, 10 mmol/L of MnCl₂, 10 mmol/L of ZnSO₄, and 2 mmol/L of each of CoCl₂, NiCl₂, Na₂Mo₄, Na₂SeO₃, and H₃BO₃.

BHISG medium: 37 g/L of a brain heart infusion powder and 10 g/L of glucose.

GAP medium: 70 g/L of glucose, 40 g/L of ammonium sulfate, 1 g/L of potassium dihydrogen phosphate, 0.4 g/L of magnesium sulfate heptahydrate, 0.01 g/L of ferrous sulfate, 0.01 g/L of manganese sulfate, 4 μg/L of biotin, 200 μg/L of VB1, and 50 g/L of calcium carbonate. The GAP medium is adjusted with KOH to a pH of 8.0 and sterilized at 115° C. for 30 min.

MS medium: 20 g/L of mannitol, 20 g/L of a soybean powder, and 15 g/L of agar.

TSB medium: 17 g/L of casein peptone, 3 g/L of soy peptone, 2.5 g/L of glucose, 5 g/L of NaCl, and 2.5 g/L of K₂HPO₄.

Primers involved in the following embodiments are shown in Table 1.

TABLE 1

| Primer name | Sequence | No. |
|---|---|---|
| pCDF-I-F1 | Gaattcgagctcggcgcgcc | SEQ ID NO: 5 |
| pCDF-I-R1 | GCTAGTCTCCTGCAAGGTCATggatcctggctgtggtgatg | SEQ ID NO: 6 |
| pCDF-YZ-F1 | Tgtccgggatctcgacgctc | SEQ ID NO: 7 |
| pCDF-YZ-R1 | Aagcattatgcggccgcaag | SEQ ID NO: 8 |
| pCDF-I-F2 | Gcgatcgctgacgtcggtac | SEQ ID NO: 9 |
| pCDF-I-R2 | Ggccggccgatatccaattg | SEQ ID NO: 10 |
| pCDF-YZ-F2 | Agtcgaacagaaagtaatcg | SEQ ID NO: 11 |
| pCDF-YZ-R2 | Gacccgtttagaggccccaa | SEQ ID NO: 12 |
| pXMJ19-I-F | gaattcagcttggctgttttg | SEQ ID NO: 13 |
| pXMJ19-I-R | aagcttaattaattctgtttcctgt | SEQ ID NO: 14 |
| pXMJ19-bpsA-F | gaaacagaattaattaagcttATGACCTTGCAGGAGACTAG | SEQ ID NO: 15 |
| pXMJ19-bpsA-R | CATCTAATAACCCTCCTCCTTTACTCACCGAGAAGGTAAC | SEQ ID NO: 16 |
| pXMJ19-entD-F | TAAAGGAGGAGGGTTATTAGatgctggatgagtctttgtt | SEQ ID NO: 17 |
| pXMJ19-entD-R | caaaacagccaagctgaattctcaagtcactgcagtcgca | SEQ ID NO: 18 |
| pXMJ19-YZ-R | gttccctactctcgcatggg | SEQ ID NO: 19 |
| pXMJ19-YZ-F | tctggataatgttttttgcg | SEQ ID NO: 20 |
| pRS425-I-F | AGGTATAGCATGAGGTCGCTCggatccactagttctagagc | SEQ ID NO: 21 |
| pRS425-I-R | AGAAACATTTTGAAGCTATGctcgagggggggcccggtac | SEQ ID NO: 22 |
| pRS425-YZ-F | Tcccagtcacgacgttgtaa | SEQ ID NO: 23 |
| pRS425-YZ-R | Ttacgccaagcgcgcaatta | SEQ ID NO: 24 |
| pKCH-hyg-I-F | Tgagctcatgagcggagaacg | SEQ ID NO: 25 |
| pKCH-hyg-I-R | cagtcgatcatagcacgatc | SEQ ID NO: 26 |
| pKCH-hyg-YZ-F | gtctgacgctcagtggaacg | SEQ ID NO: 27 |
| pKCH-hyg-YZ-R | tcatatctcattgcccccgg | SEQ ID NO: 28 |
| pKCH-YZ-F | cctcttcgctattacgccag | SEQ ID NO: 29 |
| pKCH-YZ-R | gagcggataacaatttcaca | SEQ ID NO: 30 |

Information of the strains involved in the following embodiments is shown in Table 2.

TABLE 2

| Strain name | Strain information |
|---|---|
| HG-N-Idg01 | BL21(DE3)/ pCDF-bpsA-entD |
| HG-N-Idg02 | BL21(DE3)/ pCDF-indB-indC |
| HG-N-Idg03 | ATCC13032/pXMJ19-bpsA-entD |
| HG-N-Idg04 | INVSc1/pRS425-bpsA-entD |
| HG-N-Idg05 | S. lividans TK24/pKCH-PkasO-bpsA-entD |

In the present application, the coding gene bpsA for the indigoidine synthetase is derived from S. lavendulae, the coding gene indC for the indigoidine synthetase is derived from S. chromofuscus ATCC49982, the coding gene EntD for the 4′-phosphopantetheinyl transferase is derived from C. glutamicum, and the coding gene indB for the 4′-phosphopantetheinyl transferase is derived from S. chromofuscus ATCC49982.

The coding gene bpsA for the indigoidine synthetase has a nucleotide sequence set forth in SEQ ID NO: 1, the coding gene indC for the indigoidine synthetase has a nucleotide sequence set forth in SEQ ID NO: 2, the coding gene EntD for the 4′-phosphopantetheinyl transferase has a nucleotide sequence set forth in SEQ ID NO: 3, and the coding gene indB for the 4′-phosphopantetheinyl transferase has a nucleotide sequence set forth in SEQ ID NO: 4.

In the following embodiments, a sample is detected by high-performance liquid chromatography (HPLC) as follows:

### Chromatographic Conditions

Mobile phase: methanol and pure water. Gradient elution as shown in Table 3:

TABLE 3

| Time/min | A (pure water)% | B (methanol)% |
|---|---|---|
| 0 | 80 | 20 |
| 9 | 50 | 50 |
| 13 | 80 | 20 |
| 18 | 80 | 20 |

Wavelength: 600 nm. Flow rate: 1.0 mL/min. Sample solvent: DMSO. Injection volume: 10 μL. Column temperature: 35° C. Running time: 18 min.

Chromatographic column: Galasil® EF-C18M 4.6 mm id×250 mm L (SN B06211801).

Example 1 Construction of a Strain HG-N-Idg01

A construction process of the strain HG-N-Idg01 included the following steps:

### Step 1 Construction of the Plasmid pCDF-bpsA-entD

(1) The coding gene bpsA for the indigoidine synthetase (having a nucleotide sequence set forth in SEQ ID NO: 1) and the coding gene entD for the 4′-phosphopantetheinyl transferase (having a nucleotide sequence set forth in SEQ ID NO: 3) each were synthesized by Tsingke Biotechnology Co., Ltd.
(2) The PCR amplification was conducted using primers pCDF-I-F1 and pCDF-I-R1 with a commercial plasmid pCDFDuet-1 purchased from the market as a template, and a product was purified to produce a linearized vector pCDFDuet-1.
(3) The linearized vector pCDFDuet-1 and the gene bpsA (homologous arms matching the vector had been added to two ends of the gene during the synthesis of the gene, respectively) were ligated with a seamless cloning kit. A ligation product was transformed into E. coli DH5α through chemical transformation. After a recovery culture, transformed cells were coated on an LB solid medium plate including 50 μg/mL of streptomycin, and cultured in a 37° C. incubator for about 16 h.
(4) The colony PCR was conducted using primers pCDF-YZ-F1 and pCDF-YZ-R1 for verification. A correct strain verified by PCR was cultured, and a recombinant plasmid was extracted and sent to Tsingke Biotechnology Co., Ltd. for sequencing. If having a correct sequence, the recombinant plasmid was a plasmid pCDF-bpsA.
(5) The PCR amplification was conducted using pCDF-I-F2 and pCDF-I-R2 with the plasmid pCDF-bpsA as a template, and a product was purified to produce a linearized vector pCDF-bpsA. The linearized vector pCDF-bpsA and the coding gene entD fragment for the 4′-phosphopantetheinyl transferase were ligated with a seamless cloning kit, and a ligation product was transformed into E. coli DH5a through chemical transformation. After a recovery culture, transformed cells were coated on an LB solid medium plate including 50 μg/mL of streptomycin, and cultured in a 37° C. incubator for about 16 h.
(6) The colony PCR was conducted using primers pCDF-YZ-F2 and pCDF-YZ-R2 for verification. A correct strain verified by PCR was cultured, and a recombinant plasmid was extracted and sent to Tsingke Biotechnology Co., Ltd. for sequencing. If having a correct sequence, the recombinant plasmid was the plasmid pCDF-bpsA-entD (having a sequence set forth in SEQ ID NO: 31).

### Step 2 Construction of a Strain HG-N-Idg01

(1) Competent cells produced by chemical transformation of the strain BL21 (DE3) were purchased from Qingke Biotechnology Co., Ltd., with Item No. TSC-C14.
(2) The plasmid pCDF-bpsA-entD was transformed into E. coli BL21 (DE3) through chemical transformation. After a recovery culture, transformed cells were coated on an LB solid medium plate including 50 μg/mL of streptomycin, and cultured in a 37° C. incubator for about 16 h.
(3) Single colonies grown on the LB solid medium plate were recombinant E. coli BL21 (DE3)/pCDF-bpsA-entD, which was named HG-N-Idg01.

### Example 2 Preparation of a Recombinant E. coli Cell Catalyst

(1) Single colonies of the recombinant E. coli HG-N-Idg01 were picked and transferred to 5 mL of an LB liquid medium including 50 μg/mL of streptomycin, and cultured at 37° C. and 220 rpm for 8 h.
(2) A resulting culture was inoculated at an inoculum size of 1% into 50 mL of a ZYM fermentation medium including streptomycin at 50 μg/mL and IPTG at a final concentration of 0.1 mM, and cultured at 30° C. and 220 rpm for 16 h.
(3) After the culture was completed, centrifugation was conducted at 4,000 rpm for 10 min to collect cells, which were the cell catalyst.

### Example 3 Catalytic Synthesis of N-acetyl-indigoidine with Different Substrates

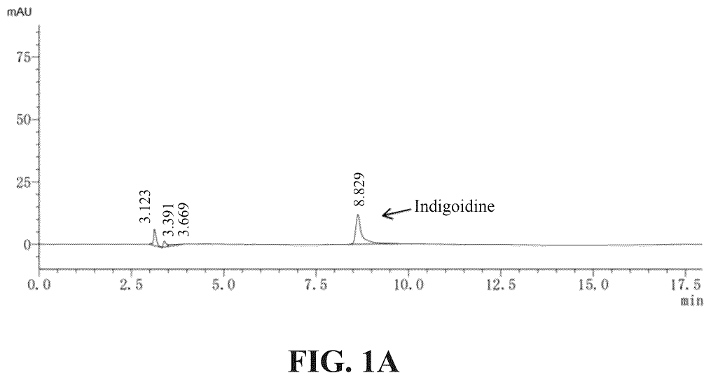

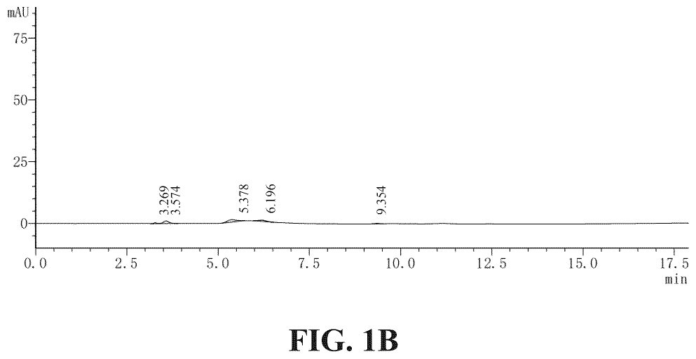

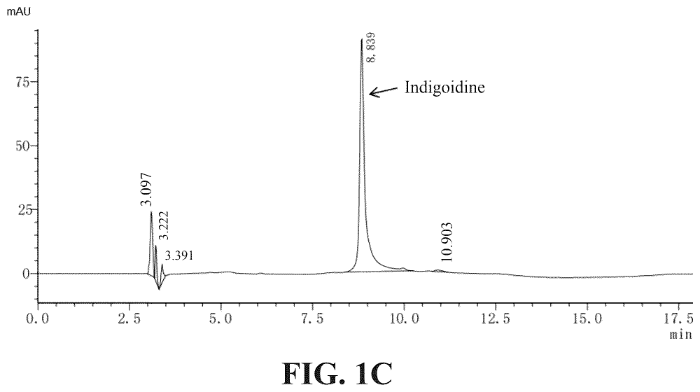

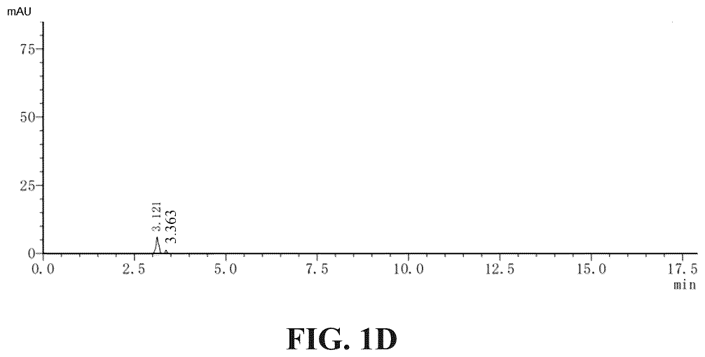

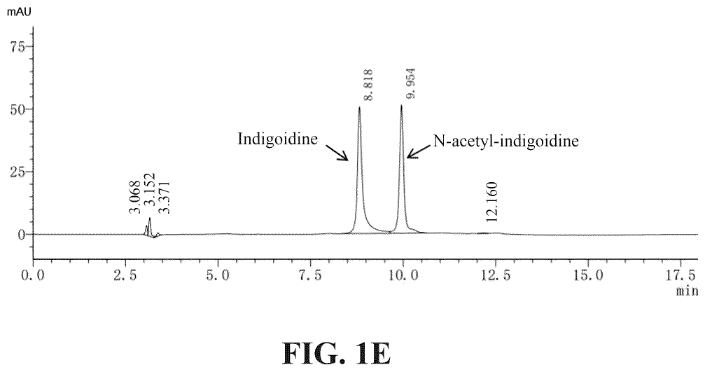

The following catalytic experiments were carried out with the cells HG-N-Idg01 obtained in Example 1 as a cell catalyst. Results were shown in FIG. 1A to FIG. 1E.

Experimental group 1:2.7 mg of an indigoidine standard was taken and added to a 25 mL volumetric flask, a DMSO solution was added, and an ultrasonic treatment was conducted for 15 min to allow full dissolution to produce a solution. The solution was cooled, diluted to a specified scale, and tested by HPLC. Test results were shown in FIG. 1A.

Experimental group 2:50 mL of a fermentation broth of the original E. coli BL21 (DE3) cultured according to the method in Example 2 was collected and centrifuged to produce a cell pellet. The cell pellet was resuspended with 10 mL of a catalytic solution (including 1 g/L of glutamine, 1 g/L of N-acetylglutamine, and 50 mM of a phosphate buffer (PB)), and a reaction was allowed at 25° C. and 220 rpm for 6 h. 100 μL of a sample was collected and tested by HPLC. Test results were shown in FIG. 1B.

Experimental group 3:50 mL of a fermentation broth of the strain HG-N-Idg01 cultured according to the method in Example 2 was collected and centrifuged to produce a cell pellet. The cell pellet was resuspended with 10 mL of a catalytic solution (including 2 g/L of glutamine and 50 mM of a phosphate buffer), and a reaction was allowed at 25° C. and 220 rpm for 6 h. 100 μL of a sample was collected and tested by HPLC. Test results were shown in FIG. 1C.

Experimental group 4:50 mL of a fermentation broth of the strain HG-N-Idg01 cultured according to the method in Example 2 was collected and centrifuged to produce a cell pellet. The cell pellet was resuspended with 10 mL of a catalytic solution (including 2 g/L of N-acetylglutamine and 50 mM of a phosphate buffer), and a reaction was allowed at 25° C. and 220 rpm for 6 h. 100 μL of a sample was collected and tested by HPLC. Test results were shown in FIG. 1D.

Experimental group 5:50 mL of a fermentation broth of the strain HG-N-Idg01 cultured according to the method in Example 2 was collected and centrifuged to produce a cell pellet. The cell pellet was resuspended with 10 mL of a catalytic solution (including 1 g/L of glutaminc, 1 g/L of N-acetylglutamine, and 50 mM of a phosphate buffer), and a reaction was allowed at 25° C. and 220 rpm for 6 h. 100 μL of a sample was collected and tested by HPLC. Test results were shown in FIG. 1E.

The results were shown in FIG. 1A to FIG. 1E. One peak of the indigoidine standard appeared at about 8.8 min in FIG. 1A. There was no significant product peak in FIG. 1B. In FIG. 1C, one peak appeared at about 8.8 min, which was consistent with the peak time of the indigoidine standard. It indicated that a catalytic product was indigoidine. There was no significant product peak in FIG. 1D. In FIG. 1E, there were one peak at about 8.8 min and one peak at about 9.9 min, which were inferred to be indigoidine and N-acetyl-indigoidine, respectively. The results show that the original strain BL21 (DE3) cannot catalyze the synthesis of N-acetyl-indigoidine from 1 g/L of glutamine and 1 g/L of N-acetylglutamine, the recombinant E. coli HG-N-Idg01 can catalyze the synthesis of N-acetyl-indigoidine from a mixture of glutamine and N-acetylglutamine through a plasmid overexpressing the genes bpsA and entD, and neither glutamine or N-acetylglutamine alone as a substrate can allow the synthesis of N-acetyl-indigoidine.

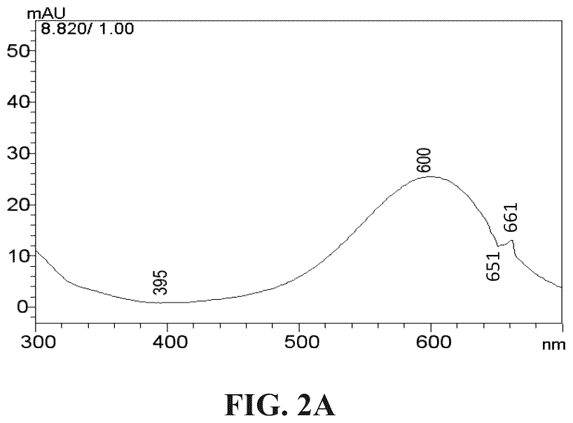

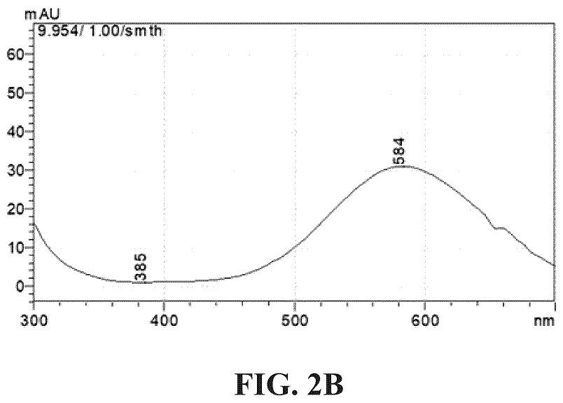

FIG. 2A and FIG. 2B show the wavelengths corresponding to the maximum absorption peaks of the experimental group 5. The wavelength corresponding to the absorption peak of indigoidine at 8.8 min is 600 nm, as shown in FIG. 2A. The wavelength corresponding to the absorption peak of N-acetyl-indigoidine at 9.9 min is 584 nm, as shown in FIG. 2B.

### Example 4 Identification of a Structure of an N-acetyl-indigoidine Sample

A catalytic reaction solution of the experimental group 5 in Example 3 was collected and centrifuged at 10,000 g for 5 min, and a resulting supernatant was discarded. A resulting cell pellet was resuspended with 10 mL of DMF and subjected to ultrasonic disruption, such that N-acetyl-indigoidine was extracted into the solvent. Centrifugation was conducted once again to produce a supernatant, and the organic solvent was evaporated under vacuum from the supernatant to produce a dark-colored solid. Then the dark-colored solid was washed twice with 10 mL of each of pure water, methanol, ethyl acetate, and hexane, and finally vacuum-lyophilized to produce 0.14 g of an N-acetyl-indigoidine powder. The above sample was sent to the Nanjing Normal University Center for Analysis and Testing for LC-MS and NMR detection.

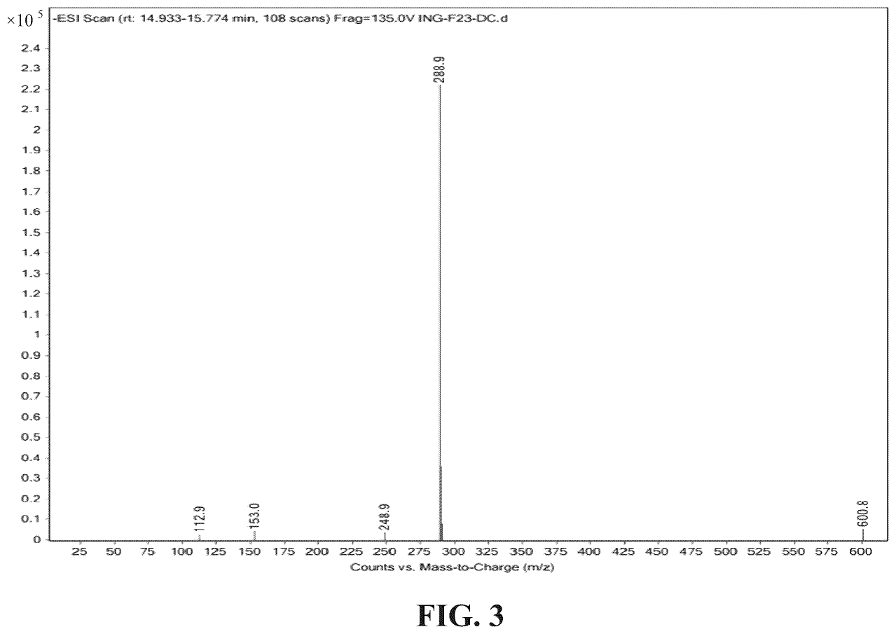

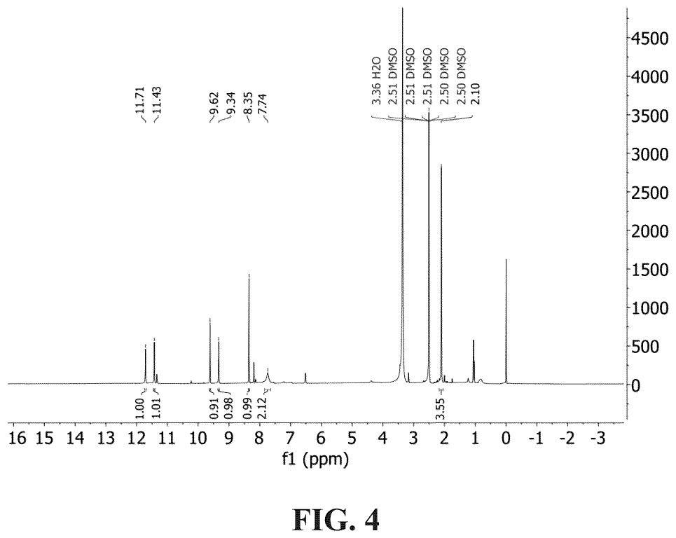

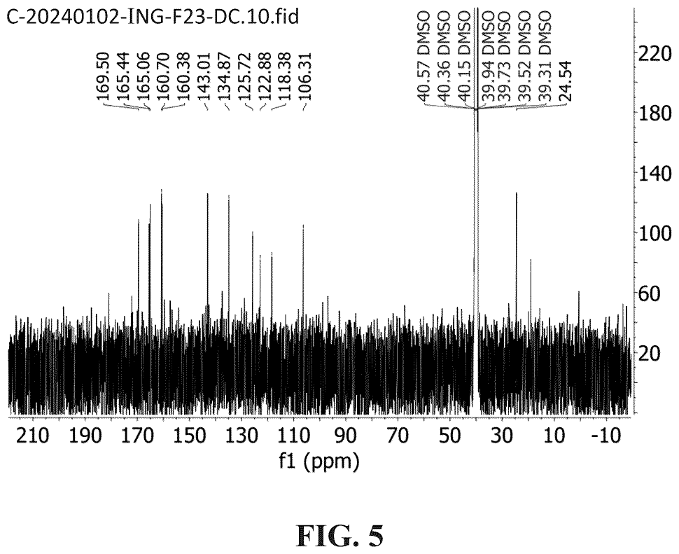

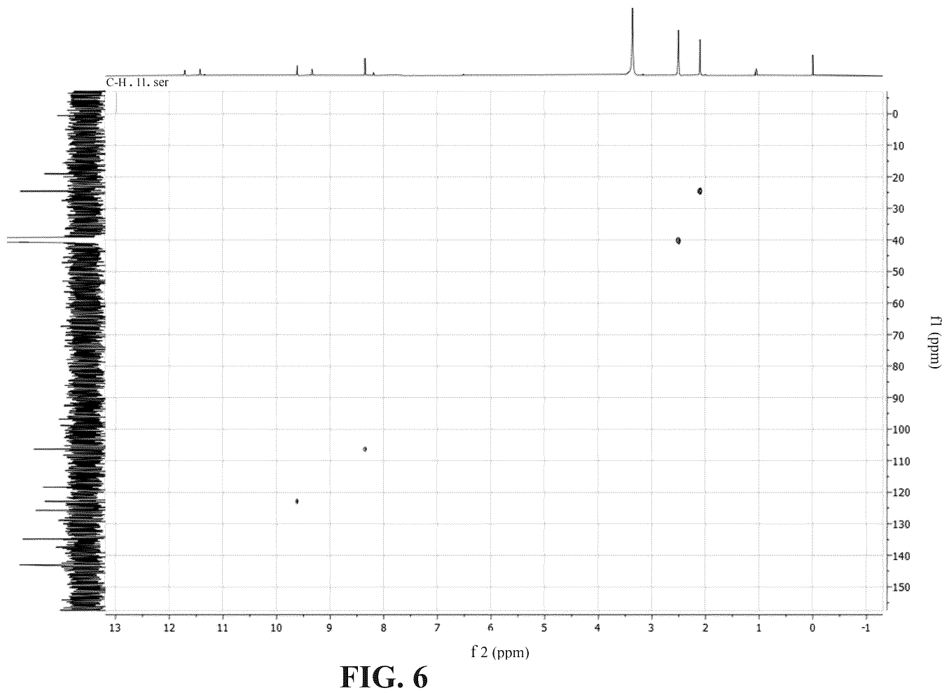

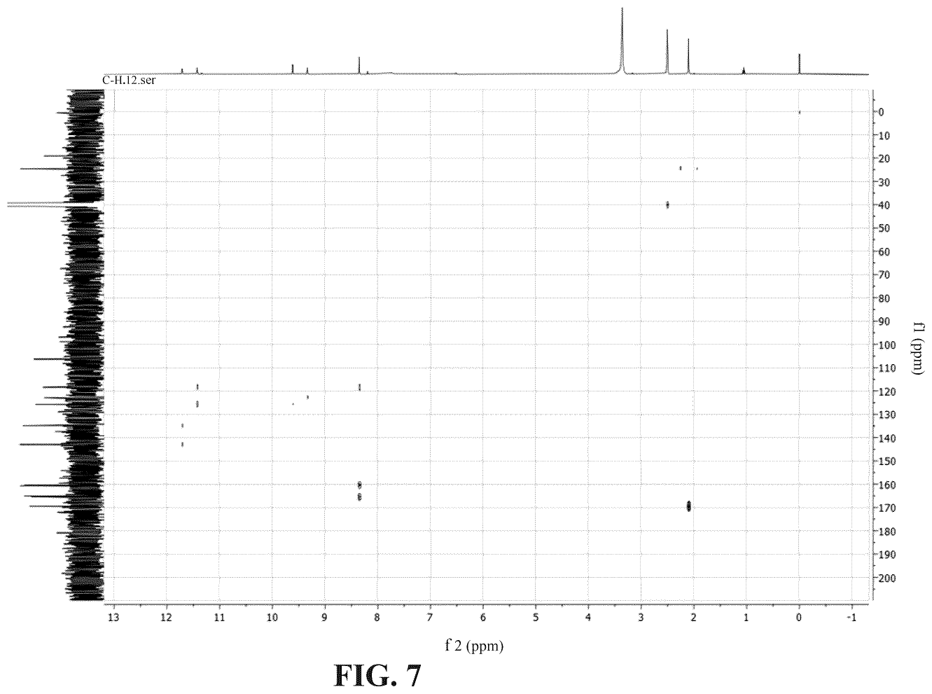

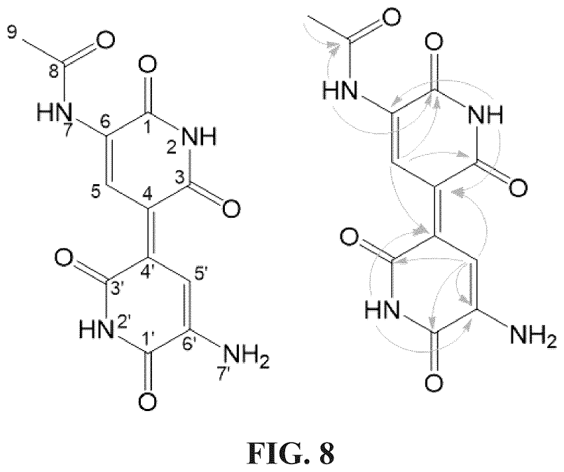

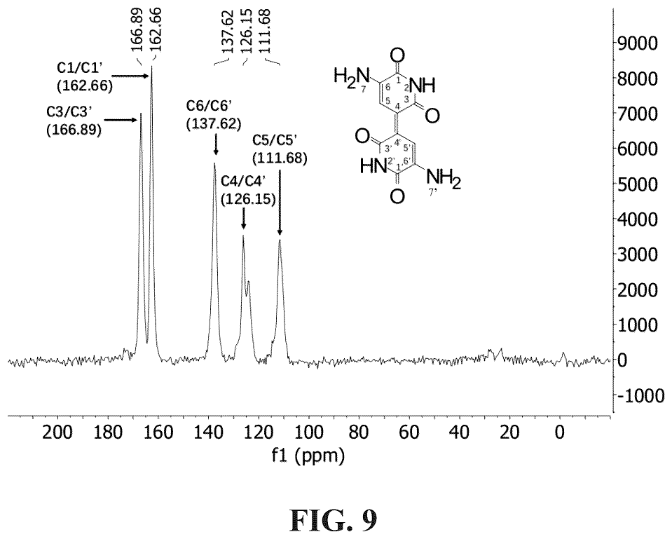

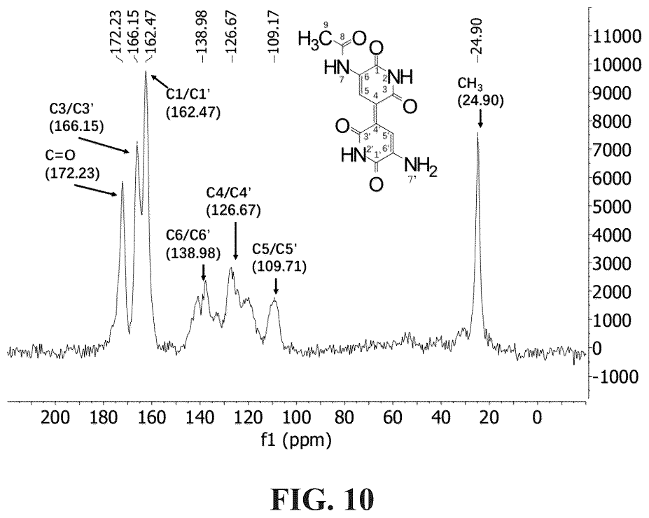

FIG. 3 shows an LC-MS spectrum of N-acetyl-indigoidine. FIG. 4 shows an ¹H-NMR spectrum of N-acetyl-indigoidine. FIG. 5 shows a ¹³C-NMR spectrum of N-acetyl-indigoidine. FIG. 6 and FIG. 7 show HSQC and HMBC NMR spectra of N-acetyl-indigoidine, respectively. FIG. 8 shows an inferred structure of N-acetyl-indigoidine. FIG. 9 shows a CPMAS NMR spectrum of indigoidine. FIG. 10 shows a CPMAS NMR spectrum of N-acetyl-indigoidine.

Table 4 shows the correspondence of data from ¹H-NMR (FIGS. 4) and ¹³C-NMR (FIG. 5) to positions of elements in the inferred structure (FIG. 8).

TABLE 4
NMR data analysis

| | | |
|---|---|---|
| Position | δ_{H} | δ_{C} |

| | | | |
|---|---|---|---|
| 1 | | | 160.38 |
| 2-NH | 11.43 | (1H, s) | |
| 3 | | | 165.44 |
| 4 | | | 134.87 |
| 5 | 8.35 | (1H, s) | 106.31 |
| 6 | | | 143.01 |
| 7-NH | 9.34 | (1H, s) | |
| 8 | | | 169.50 |
| 9 | 2.10 | (3H, s) | 24.54 |
| 1′ | | | 160.70 |
| 2′-NH | 11.71 | (1H, s) | |
| 3′ | | | 165.06 |
| 4′ | | | 118.39 |
| 5′ | 9.62 | (1H, s) | 122.88 |
| 6′ | | | 125.72 |
| 7′-NH₂ | 7.74 | (2H, s) | |

The relative molecular mass of indigoidine was 248.19 and the relative molecular mass of acetyl was 43. The LC-MS results (FIG. 3) showed that the sample sent for testing had a relative molecular mass of 289.9, which was consistent with the theoretical value of N-acetyl-indigoidine.

As shown in the ¹H-NMR spectrum (FIG. 4), a peak at about 8 2.50 ppm was a peak of the solvent DMSO and a peak at 8 3.36 ppm was a peak of water. An isolated peak at 11.71 ppm was used as an integration reference, which was set to 1. According to the HSQC spectrum (FIG. 6), there was no point corresponding to 11.71 ppm on the abscissa axis, indicating hydrogen bonded with nitrogen. There were also no points corresponding to 11.43 ppm, 7.74 ppm, and 9.34 ppm, indicating hydrogen bonded with nitrogen. Peaks at 8.35 ppm and 9.62 ppm were singlets, and there was carbon corresponding to 100 ppm to 160 ppm on the HSQC spectrum (FIG. 6), indicating olefinic carbon. A peak at 2.10 ppm was an s peak with an integral of 3, indicating the presence of one methyl group. An integral of the peak at 7.74 ppm was 2, indicating a primary amine. Integrals of the peaks at 11.71 ppm, 11.43 ppm, and 9.34 ppm were 1, indicating a secondary amine. It could be inferred through the ¹H-NMR that the compound had one primary amine, three secondary amines, two double bonds, and one methyl group.

In combination with the analysis of the ¹³C-NMR spectrum (FIG. 5), a peak at 8 24.54 ppm was a methyl signal, and peaks at δ 160 ppm to 180 ppm indicated carbonyl signals, including five carbonyl groups at δ 169.50 ppm, 165.44 ppm, 165.06 ppm, 160.70 ppm, and 160.38 ppm, respectively. Generally, a range of 100 ppm to 150 ppm corresponded to sp² hybridized carbon atoms. Peaks at 122.88 ppm and 106.31 ppm were correlated with carbons corresponding to the ¹H-NMR spectrum, indicating olefinic carbon. The remaining peaks at 143.01 ppm, 134.87 ppm, 125.72 ppm, and 118.39 ppm all were attributed to unsaturated carbons. In summary, with reference to the structural formula of indigoidine, it was inferred that a structural formula of N-acetyl-indigoidine was shown in FIG. 8. The attribution of each signal of the ¹H-NMR spectrum and the ¹³C-NMR spectrum was shown in Table 4.

It was inferred that a molecular formula of this new substance was C₁₂H₁₀N₄O₅. It could be seen from the comparison of CPMAS NMR spectra of indigoidine and N-acetyl-indigoidine (FIG. 9 and FIG. 10) that characteristic peaks of —C═O and —CH₃ in acetyl appeared at δ=172.23 ppm and δ=24.90 ppm, respectively. Thus, this new substance was named N-acetyl-indigoidine. This structure had not been documented and was a completely new substance. This new substance was dark-blue in a solution, and the value of its maximum light absorption was 584 nm. Therefore, this new substance was inferred to be a novel blue pigment.

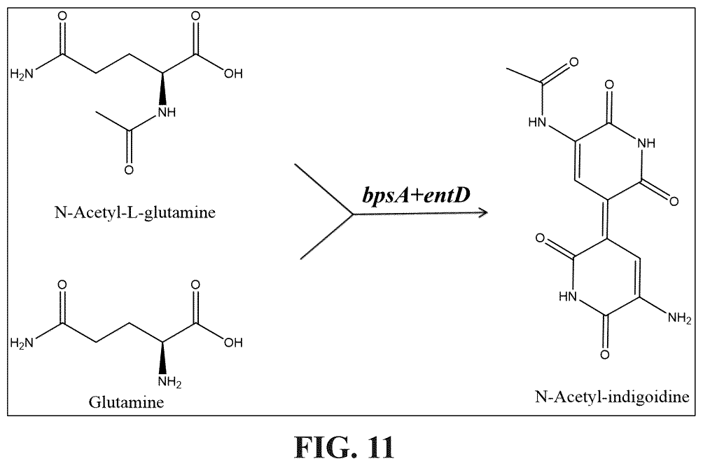

A biosynthetic route of N-acetyl-indigoidine by the strain HG-N-Idg01 was shown in FIG. 11.

### Example 5 Construction of a Strain HG-N-Idg02

In order to compare the biological activities of indigoidine synthetases and phosphopantetheinyl transferases from different species, a recombinant E. coli strain HG-N-Idg02 overexpressing a coding gene (indC) for an indigoidine synthetase and a coding gene (indB) for a 4′-phosphopantetheinyl transferase that were derived from S. chromofuscus ATCC49982 was constructed in this example.

A construction process of the recombinant E. coli strain HG-N-Idg02 was as follows:

### Step 1 Construction of a Plasmid pCDF-indB-indC

(1) The coding gene indC for the indigoidine synthetase (having a nucleotide sequence set forth in SEQ ID NO: 3) and the coding gene indB for the 4′-phosphopantetheinyl transferase (having a nucleotide sequence set forth in SEQ ID NO: 4) each were synthesized by Tsingke Biotechnology Co., Ltd.
(2) The plasmid pCDF-indB-indC was constructed according to the method in Example 1.

### Step 2 Construction of a Strain HG-N-Idg02

The pCDF-indB-indC constructed in the step 1 was transformed into BL21 (DE3) through chemical transformation to produce recombinant E. coli BL21 (DE3)/pCDF-indB-indC, which was named HG-N-Idg02.

### Example 6 Comparison of Catalytic Abilities of Indigoidine Synthetases From Different Sources

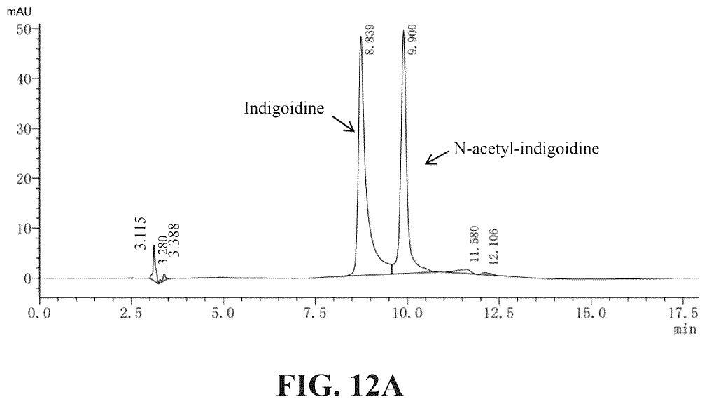

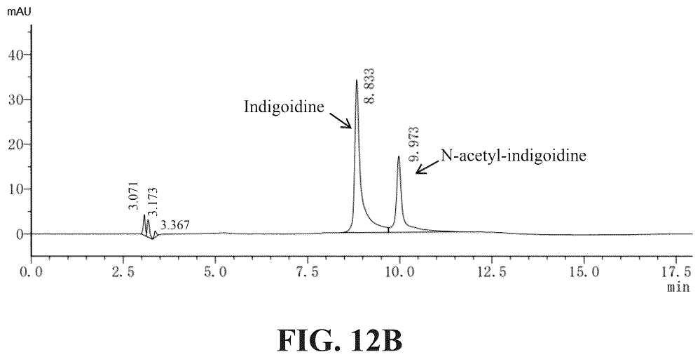

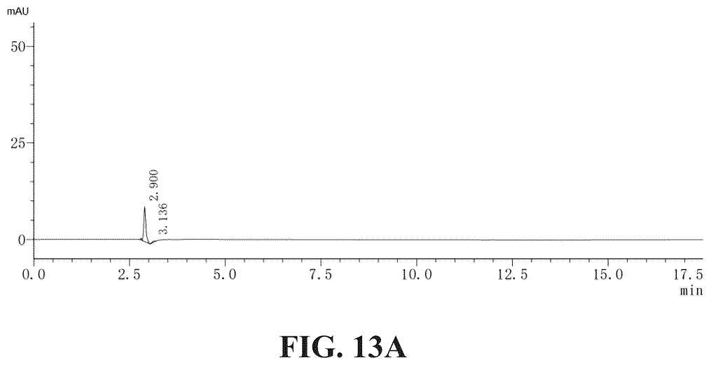

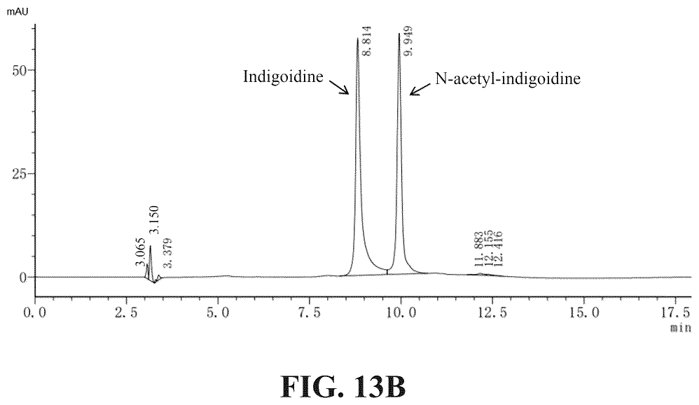

Cell catalysts HG-N-Idg01 and HG-N-Idg02 each were prepared by the culture method in Example 2. Cells were resuspended with 10 mL of a catalytic solution (1 g/L of glutamine+1 g/L of N-acetylglutamine +50 mM of PB), and a reaction was allowed at 20° C. and 220 rpm for 6 h. 100 μL of a resulting reaction solution was collected for HPLC detection. Test results were shown in FIG. 13A and FIG. 12B. Catalytic reaction solutions of HG-N-Idg01 and HG-N-Idg02 both presented two peaks at 8.8 min and 9.9 min, respectively. A catalytic product of HG-N-Idg01 had larger peak areas of both indigoidine and N-acetyl-indigoidine than a catalytic product of HG-N-Idg02. The results showed that both indB and indC could catalyze the production of indigoidine and N-acetyl-indigoidine from glutamine and N-acetylglutamine, but exhibited lower enzyme activities than bpsA and entD.

In the present application, the biosynthesis of N-acetyl-indigoidine was achieved in E. coli with indigoidine synthetases and phosphopantetheinyl transferases from different sources.

### Example 7 Construction of Recombinant C. glutamicum HG-N-Idg03

In this example, a construction process of the recombinant C. glutamicum HG-N-Idg03 was provided, including the following steps:

### Step 1 Construction of a Plasmid pXMJ19-bpsA-entD

(1) With the plasmid pCDF-bpsA-entD constructed in Example 1 as a template, the PCR amplification was conducted using primers pXMJ19-bpsA-F and pXMJ19-bpsA-R to produce a gene fragment bpsA, and the PCR amplification was conducted using primers pXMJ19-entD-F and pXMJ19-entD-R to produce a gene fragment entD.
(2) With a commercial plasmid pXMJ19 purchased from the market as a template, the PCR amplification was conducted using primers pXMJ19-I-F and pXMJ19-I-R to produce a lincarized vector pXMJ19.
(3) The ligation was conducted with a seamless cloning kit of Takara Bio. A ligation product was chemically transformed into E. coli DH5α. After a recovery culture, transformed cells were coated on an LB solid medium plate including 15 μg/mL of chloramphenicol, and cultured in a 37° C. incubator for about 16 h.
(4) The colony PCR was conducted using primers pXMJ19-YZ-F and pXMJ19-YZ-R for verification. A correct strain verified by PCR was cultured, and a recombinant plasmid was extracted and sent to Tsingke Biotechnology Co., Ltd. for sequencing. If having a correct sequence, the recombinant plasmid was the plasmid pXMJ19-bpsA-entD.

### Step 2 Preparation of ATCC13032 Electrocompetent Cells

(1) Single colonies of ATCC13032 were picked and inoculated in 5 mL of an antibiotic-free BHISG medium, and cultured overnight at 30° C. and 220 rpm.
(2) After OD₆₀₀ was determined, a bacterial solution of 15 OD (15 mL of a fermentation broth with OD₆₀₀=1 or 7.5 mL of a fermentation broth with OD₆₀₀=2) was inoculated in 50 mL of a BHISGGT medium (including 0.1% of Tween 80 and 50 μg/L of biotin), cultured at 30° C. and 220 rpm until OD₆₀₀ was about 1.0, and then taken out and incubated in an ice bath for 20 min.
(3) A resulting bacterial solution was transferred to a 50 mL centrifuge tube and centrifuged at 4° C. and 2,600×g for 10 min, a resulting supernatant was discarded, and the residual liquid was removed by a pipette.
(4) A resulting cell pellet was resuspended with 50 mL of 10% glycerol (pre-cooled) and centrifuged at 4° C. and 2,600×g for 10 min, a resulting supernatant was discarded, and the residual liquid was removed by a pipette. This step was repeated once.
(5) Resulting cells were resuspended with 100 μL of 10% glycerol (pre-cooled) and transferred to a 1.5 mL EP tube for later use.

### Step 3 Construction of the Recombinant Strain HG-N-Idg03

(1) 200 ng of the plasmid pXMJ19-bpsA-entD was taken and added to the electrocompetent cells prepared in the step 2, mixing was conducted gently, and a resulting mixture was added to an electroporation cuvette and incubated in an ice bath for 5 min to 10 min.
(2) After an electric shock was conducted 2 times to 3 times at 1.8 KV, a BHISG medium pre-heated at 46° C. was added immediately, and a heat shock was conducted in a 46° C. water bath for 6 min. A transformation solution produced after the heat shock was incubated in a 30° C. shaker for 2 h.
(3) 1 mL of a transformation solution produced after the incubation was taken and centrifuged at 4,000 rpm, and most of a resulting supernatant was discarded. The residual cell pellet was resuspended, coated on a BHISG plate including chloramphenicol, and cultured in a 30° C. incubator for 48 h.
(4) Single colonies grown were the recombinant C. glutamicum HG-N-Idg03.

### Example 8 Catalytic Synthesis of N-acetyl-indigoidine by the Recombinant C. glutamicum HG-N-Idg03

Single colonies of the recombinant C. glutamicum strain HG-N-Idg03 were picked and transferred to 5 mL of a BHISG medium including 15 μg/mL of chloramphenicol and 50 μg/L of biotin, and cultured at 30° C. and 220 rpm for 16 h. A resulting culture was inoculated into 50 mL of a GAP medium at an inoculum size of 1%, chloramphenicol was added at a final concentration of 15 μg/mL and biotin was added at a final concentration of 50 μg/L, and a culture was conducted at 30° C. and 220 rpm for 3 h. Then IPTG was added at a final concentration of 1 mM, and a culture was further conducted for 24 h. Resulting cells were collected as a cell catalyst through centrifugation.

The cells were resuspended with 10 mL of a catalytic reaction solution (including 1 g/L of glutamine, 1 g/L of N-acetylglutamine, and 50 mM of PB), and a reaction was allowed at 25° C. and 220 rpm for 6 h. 100 μL of a sample was collected and tested by HPLC. The original C. glutamicum strain ATCC13032 was adopted as a control group.

Test results were shown in FIG. 13A and FIG. 13B. FIG. 13A shows the test results of the strain ATCC13032, where there is no obvious peak at 8.8 min and 9.9 min. FIG. 13B shows the test results of the strain HG-N-Idg03, where there are one peak at 8.8 min and one peak at 9.9 min, which are consistent with the peak times of indigoidine and N-acetyl-indigoidine, respectively. It indicates that the original strain ATCC13032 cannot catalyze the synthesis of N-acetyl-indigoidine from glutamine and N-acetylglutamine, and the recombinant C. glutamicum HG-N-Idg03 can catalyze the synthesis of N-acetyl-indigoidine from glutamine and N-acetylglutamine through a plasmid overexpressing the genes bpsA and entD.

### Example 9 Construction of Recombinant S. cerevisiae HG-N-Idg04

In this example, a construction process of the recombinant S. cerevisiae HG-N-Idg04 was provided, including the following steps:

### Step 1 Construction of a Plasmid pRS425-bpsA-entD

(1) A coding gene bpsA for an indigoidine synthetase-was synthesized by Tsingke Biotechnology Co., Ltd. During the synthesis, TEF1 promoter and CYC1 terminator sequences were introduced. An expression cassette of TEF1 promoter-bpsA-CYC1 terminator had a nucleotide sequence set forth in SEQ ID NO: 32.
(2) A coding gene entD for a 4′-phosphopantetheinyl transferase-was synthesized by Tsingke Biotechnology Co., Ltd. During the synthesis, TEF1 promoter and ADHI terminator sequences were introduced. An expression cassette of TEF1 promoter-entD-ADHI terminator had a nucleotide sequence set forth in SEQ ID NO: 33.
(3) With a plasmid pRS425 purchased from the market as a template, the PCR amplification was conducted using primers pRS425-I-F and pRS425-I-R to produce a linearized vector pRS425.
(4) The linearized vector pRS425, the fragment of TEF1 promoter-bpsA-CYC1 terminator, and the fragment of TEF1 promoter-entD-ADHI terminator were ligated with a seamless cloning kit. A ligation product was transformed into E. coli DH5α through chemical transformation. After a recovery culture, transformed cells were coated on an LB solid medium plate including 100 μg/mL of ampicillinum, and cultured in a 37° C. incubator for about 16 h.
(5) The colony PCR was conducted using primers pRS425-YZ-F and pRS425-YZ-R for verification. A correct strain verified by PCR was cultured, and a recombinant plasmid was extracted and sent to Tsingke Biotechnology Co., Ltd. for sequencing. If having a correct sequence, the recombinant plasmid was the plasmid pRS425-bpsA-entD.

### Step 2 Construction of a Strain HG-N-Idg04

The recombinant plasmid pRS425-bpsA-entD was transformed into S. cerevisiae INVSc1 through lithium acetate transformation. After a recovery culture, transformed cells were coated on an SC-Leu agar medium and cultured at 30° C. for 48 h. Single colonies grown were recombinant S. cerevisiae INVSc1/pRS425-bpsA-entD, which was the recombinant S. cerevisiae HG-N-Idg04.

### Example 10 Catalytic Synthesis of N-acetyl-indigoidine by the Recombinant S. cerevisiae HG-N-Idg04

Single colonies of the recombinant S. cerevisiae HG-N-Idg04 were picked and inoculated in 5 mL of an SC-Leu liquid medium, and cultured at 30° C. and 220 rpm for 24 h. A resulting culture was centrifuged at 1,500 g and 4° C. for 5 min, and a resulting supernatant was discarded. A resulting cell pellet was resuspended with 50 mL of an SC-Leu liquid medium, 10 g/L of galactose was added, and a culture was allowed at 30° C. and 220 rpm for 24 h. Resulting cells were collected through centrifugation, which were a cell catalyst HG-N-Idg04. The cells were resuspended with 10 mL of a catalytic solution (including 1 g/L of glutamine, 1 g/L of N-acetylglutamine, and 50 mM of PB), and a reaction was allowed at 25° C. and 220 rpm for 6 h. 100 μL of a sample was collected and tested by HPLC. The original strain INVSc1 was adopted as a control group.

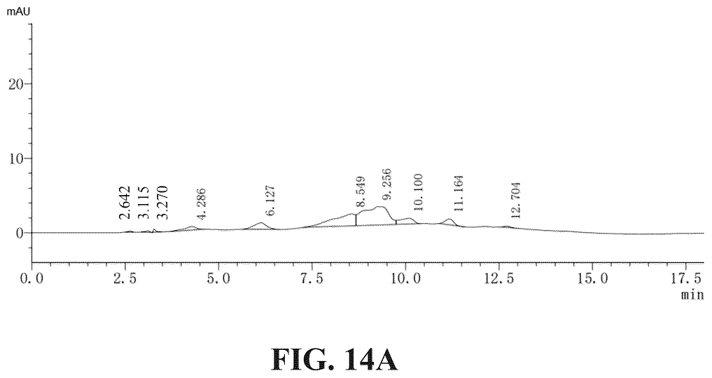

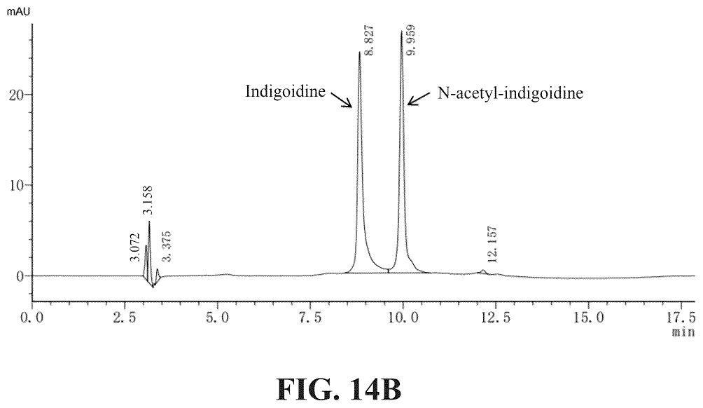

Test results were shown in FIG. 14A and FIG. 14B. FIG. 14A shows the test results of the strain INVSc1, where there is no obvious peak at 8.8 min and 9.9 min. FIG. 14B shows the test results of the strain HG-N-Idg04, where there are one peak at 8.8 min and one peak at 9.9 min, which are consistent with the peak times of indigoidine and N-acetyl-indigoidine, respectively. It indicates that the original strain INVSc1 cannot catalyze the synthesis of N-acetyl-indigoidine from glutamine and N-acetylglutamine, and the recombinant S. cerevisiae HG-N-Idg04 can catalyze the synthesis of N-acetyl-indigoidine from glutamine and N-acetylglutamine through a plasmid overexpressing the genes bpsA and entD.

### Example 11 Construction of Recombinant S. lividans HG-N-Idg05

In this example, a construction process of the recombinant S. lividans HG-N-Idg05 was provided, including the following steps:

### Step 1 Construction of a Hygromycin B Resistance Plasmid pKC-hyg

(1) With a commercial plasmid pKC1139 purchased from the market as a template, the PCR amplification was conducted using primers pKCH-hyg-I-F and pKCH-hyg-I-R to produce a lincarized vector pKC1139.
(2) A hygromycin B resistance gene (hyg') was synthesized by Tsingke Biotechnology Co., Ltd. The hygromycin B resistance gene had a nucleotide sequence set forth in SEQ ID NO: 34.
(3) The linearized vector pKC1139 and the fragment of the hygromycin B resistance gene were ligated with a seamless cloning kit of Takara Bio. A ligation product was chemically transformed into E. coli DH5α. After a recovery culture, transformed cells were coated on an LB solid medium plate including 100 μg/mL of hygromycin B, and cultured in a 37° C. incubator for about 16 h.
(4) The colony PCR was conducted using primers pKC-hyg-YZ-F and pKC-hyg-YZ-R for verification. A correct strain verified by PCR was cultured, and a recombinant plasmid was extracted and sent to Tsingke Biotechnology Co., Ltd. for sequencing. If having a correct sequence, the recombinant plasmid was the hygromycin B resistance plasmid pKC-hyg.

### Step 2 Construction of a Plasmid pKCH-PkasO-bpsA-entD

(1) With the plasmid pKC-hyg constructed in the step 1 as a template, the PCR amplification was conducted using primers pKCH-I-F and pKCH-I-R to produce a linearized vector pKCH-hyg.
(2) A gene sequence of the PkasO-bpsA-entD expression cassette was synthesized by Tsingke Biotechnology Co., Ltd. The gene sequence of the expression cassette had a nucleotide sequence set forth in SEQ ID NO: 35.
(3) The linearized vector pKCH-hyg and the PkasO-bpsA-entD were ligated with a seamless cloning kit of Takara Bio. A ligation product was transformed into E. coli DH5α. After a recovery culture, transformed cells were coated on an LB solid medium plate including 100 μg/mL of hygromycin B, and cultured in a 37° C. incubator for about 16 h.
(4) The colony PCR was conducted using primers pKCH-YZ-F and pKCH-YZ-R for verification. A correct strain verified by PCR was cultured, and a recombinant plasmid was extracted and sent to Tsingke Biotechnology Co., Ltd. for sequencing. If having a correct sequence, the recombinant plasmid was the plasmid pKCH-PkasO-bpsA-entD.

### Step 3 Construction of the Recombinant Strain HG-N-Idg05

S. lividans TK24 and E. coli ET12567/pUZ8002 were purchased from Biofeng (Shanghai, China). The recombinant plasmid pKCH-PkasO-bpsA-entD was transformed into S. lividans TK24 through conjugative transfer to construct HG-N-Idg05.

A specific experimental process was as follows:
(1) The plasmid pKCH-PkasO-bpsA-entD for conjugative transfer was transformed into E. coli ET12567/pUZ8002.
(2) Conjugative transfer between two parents was performed with E. coli ET12567/pUZ8002 carrying the plasmid and S. lividans TK24.
(3) A culture was conducted at 28° C. for 18 h, and then hygromycin B and nalidixic acid were added for covering.
(4) A culture was conducted at 28° C. for 3 d to 5 d, and resulting recipients were picked and subjected to genomic DNA extraction.
(5) The PCR verification was conducted with primers pKCH-YZ-F and pKCH-YZ-R. A correct strain verified by PCR was recombinant S. lividans

HG-N-Idg05: TK24/pKCH-PkasO-bpsA-entD.

### Example 12 Catalytic Synthesis of N-acetyl-indigoidine by the Recombinant S. lividans HG-N-Idg05

The recombinant S. lividans strain HG-N-Idg05 grown on an MS medium for 4 d to 5 d was picked by a sterile pipette tip, inoculated in a primary shake flask with 10 mL of a TSB medium, and cultured at 28° C. for 48 h. 1 mL of a resulting culture was collected and transferred to a secondary shake flask with 50 mL of a TSB medium, and cultured at 28° C. for 72 h. Resulting cells were collected through centrifugation, which were a cell catalyst HG-N-Idg05. The cells were resuspended with 10 mL of a catalytic solution (including 1 g/L of glutamine, 1 g/L of N-acetylglutamine, and 50 mM of PB), and a reaction was allowed at 25° C. and 220 rpm for 6 h. 100 μL of a sample was collected and tested by HPLC. The original S. lividans strain TK24 was adopted as a control group.

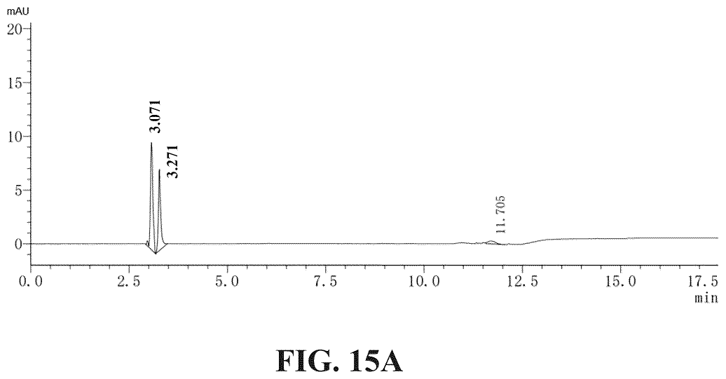

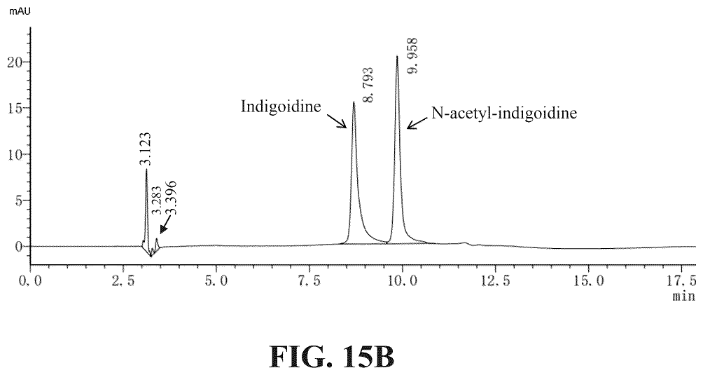

Test results were shown in FIG. 15A and FIG. 15B. FIG. 15A shows the test results of the S. lividans strain TK24, where there is no obvious peak at 8.8 min and 9.9 min. FIG. 15B shows the test results of the strain HG-N-Idg05, where there are one peak at around 8.8 min and one peak at around 9.9 min, which are consistent with the peak times of indigoidine and N-acetyl-indigoidine, respectively. It indicates that the original strain TK24 cannot catalyze the synthesis of N-acetyl-indigoidine from glutamine and N-acetylglutamine, and the recombinant S. lividans HG-N-Idg05 can catalyze the synthesis of N-acetyl-indigoidine from glutamine and N-acetylglutamine through a plasmid overexpressing the genes bpsA and entD.

The present application achieves the catalytic synthesis of N-acetyl-indigoidine from glutamine and N-acetylglutamine in E. coli, C. glutamicum, S. cerevisiae, and Streptomyces.

### Comparative Example 1 Comparison of Colors of Solutions of Indigoidine and N-acetyl-indigoidine in DMSO

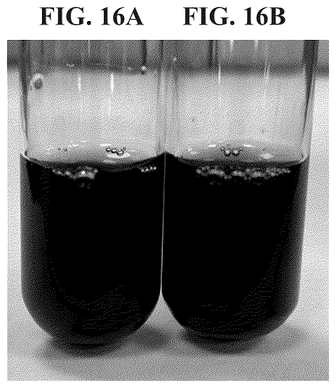

1 mg of an indigoidine standard was weighed and dissolved with 10 mL of DMSO to produce a solution of indigoidine in DMSO. 1 mg of N-acetyl-indigoidine purified in Example 4 was weighed and dissolved with 10 mL of DMSO to produce a solution of N-acetyl-indigoidine in DMSO. A color difference between the solution of indigoidine in DMSO and the solution of N-acetyl-indigoidine in DMSO was observed, and results were shown in FIG. 16A and FIG. 16B. As shown in FIG. 16A, the solution of N-acetyl-indigoidine in DMSO is bright-blue. As shown in FIG. 16B, the solution of indigoidine in DMSO is blue. The solution of N-acetyl-indigoidine in DMSO has a more vibrant and brighter color than the solution of indigoidine in DMSO.

### Comparative Example 2 Comparison of Colors of Solutions of Indigoidine and N-acetyl-indigoidine in Water

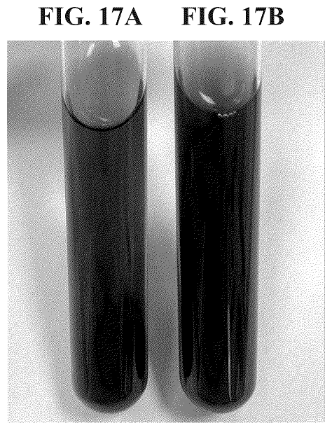

1 mg of an indigoidine standard was weighed and dissolved with 10 mL of ddH₂O to produce a solution of indigoidine in ddH₂O. 1 mg of N-acetyl-indigoidine purified in Example 4 was weighed and dissolved with 10 mL of ddH₂O to produce a solution of N-acetyl-indigoidine in ddH₂O. A color difference between the solution of indigoidine in ddH₂O and the solution of N-acetyl-indigoidine in ddH₂O was observed, and results were shown in FIG. 17A and FIG. 17B. As shown in FIG. 17A, the solution of N-acetyl-indigoidine in ddH₂O is blue. As shown in FIG. 17B, the solution of indigoidine in ddH₂O is purple-blue. It can be seen that N-acetyl-indigoidine has a more authentic color than indigoidine.

Finally, it should be noted that the above examples are provided merely to describe the technical solutions of the present application, rather than to limit the protection scope of the present application. Although the present application is described in detail with reference to preferred examples, a person of ordinary skill in the art should understand that modifications or equivalent replacements may be made to the technical solutions of the present application without departing from the spirit and scope of the technical solutions of the present application.

1. A blue pigment, wherein the blue pigment has a chemical name of N-acetyl-indigoidine, a molecular formula of C₁₂H₁₀N₄O₅, and a chemical structure set forth in the following formula I:

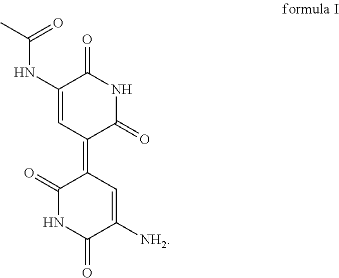 [SMILES: CC(=O)NC1=C/C(=C2/C=C(N)C(=O)NC2=O)C(=O)NC1=O]

2. A biosynthesis method of the blue pigment according to claim 1, comprising: expressing an indigoidine synthetase and a 4′-phosphopantetheinyl transferase by a metabolically engineered strain to catalyze biosynthesis of the blue pigment from glutamine and N-acetylglutamine.

3. The biosynthesis method of the blue pigment according to claim 2, wherein a coding gene for the indigoidine synthetase comprises one selected from the group consisting of a coding gene bpsA for the indigoidine synthetase and a coding gene indC for the indigoidine synthetase; and
a coding gene for the 4′-phosphopantetheinyl transferase comprises one selected from the group consisting of a coding gene EntD for the 4′-phosphopantetheinyl transferase and a coding gene indB for the 4′-phosphopantetheinyl transferase.

4. The biosynthesis method of the blue pigment according to claim 2, wherein a construction process of the metabolically engineered strain comprises:
introducing a coding gene bpsA for the indigoidine synthetase- and a coding gene EntD for the 4′-phosphopantetheinyl transferase into a host strain to construct the metabolically engineered strain; or
introducing a coding gene indC for the indigoidine synthetase and a coding gene indB for the 4′-phosphopantetheinyl transferase into the host strain to construct the metabolically engineered strain.

5. The biosynthesis method of the blue pigment according to claim 4, wherein the construction process of the metabolically engineered strain comprises the following steps:
amplifying the coding gene bpsA for the indigoidine synthetase and the coding gene EntD for the 4′-phosphopantetheinyl transferase separately through polymerase chain reaction (PCR), and ligating amplified fragments to a plasmid to produce the metabolically engineered strain; or
amplifying the coding gene indC for the indigoidine synthetase and the coding gene indB for the 4′-phosphopantetheinyl transferase separately through PCR, and ligating amplified fragments to the plasmid to produce the metabolically engineered strain.

6. The biosynthesis method of the blue pigment according to claim 5, wherein the plasmid comprises at least one selected from the group consisting of pCDFDuet-1, pXMJ19, pRS425, and pKC1139 plasmids.

7. The biosynthesis method of the blue pigment according to claim 5, wherein the PCR amplification is conducted using primers having sequences set forth in SEQ ID NOs: 5-30.

8. The biosynthesis method of the blue pigment according to claim 4, wherein the host strain comprises at least one selected from the group consisting of Escherichia coli (E. coli), Corynebacterium glutamicum (C. glutamicum), Saccharomyces cerevisiae (S. cerevisiae), and Streptomyces lividans (S. lividans).

9. The biosynthesis method of the blue pigment according to claim 4, comprising:
collecting cells produced after an induction culture of the metabolically engineered strain through centrifugation;
resuspending the cells with a catalytic solution comprising phosphate buffered saline, glutamine, and N-acetylglutamine;
allowing catalysis to produce a conversion solution; and
collecting the blue pigment in the conversion solution through centrifugation.

10. The biosynthesis method of the blue pigment according to claim 9, wherein in the catalytic solution, a concentration of the glutamine is 0.1 g/L to 10 g/L and a concentration of the N-acetylglutamine is 0.1 g/L to 10 g/L.

11. A cell catalyst comprising the metabolically engineered strain according to claim 4.

12. A cell catalyst comprising the metabolically engineered strain according to claim 5.
